(19) United States
(12) Patent Application Publication
(10) Pub. No.: US 2025/0268997 A1
(43) Pub. Date: Aug. 28, 2025

(54) A Subunit Cryptococcus Vaccine

(71) Applicant: University of Massachusetts, Boston, MA (US)

(72) Inventors: Stuart Levitz, Wayland, MA (US); Charles A. Specht, Littleton, MA (US)

(21) Appl. No.: 18/705,007

(22) PCT Filed: Oct. 28, 2022
(86) PCT No.: PCT/US2022/048142

§ 371 Date: Apr. 26, 2024

Related U.S. Application Data
(60) Provisional application No. 63/284,335, filed on Nov. 30, 2021.
(60) Provisional application No. 63/273,628, filed on Oct. 29, 2021.

(51) Int. Cl. A61K 39/00; A61P 31/10

(52) U.S. Cl. CPC A61K 39/0002; A61P 31/10; A61K 2039/55555; A61K 2039/575

(57) ABSTRACT

Provided herein are subunit vaccines against Cryptococcus.

### CLAIM OF PRIORITY

This application claims the benefit of U.S. Provisional Patent Application Ser. No. 63/284,335, filed on Nov. 30, 2021, and Application Ser. No. 63/273,628, filed on Oct. 29, 2021, the entire contents of which are hereby incorporated by reference in their entirety.

### FEDERALLY SPONSORED RESEARCH OR DEVELOPMENT

This invention was made with Government support under Grant Nos. AI025780 and AI125045 awarded by the National Institutes of Health and Contract No. 75N93019C00064 awarded by the National Institute of Allergy and Infectious Diseases. The Government has certain rights in the invention.

### TECHNICAL FIELD

The disclosure relates generally to vaccines against cryptococcosis. More specifically, it relates to recombinant multivalent subunit vaccines. The disclosure further relates to the making and use of these vaccines for raising an immune response for protecting a subject against cryptococcosis.

### BACKGROUND

Cryptococcosis is a potentially fatal fungal infection that can affect the lungs or brain. In the lungs, it presents as a pneumonia; in the brain, it appears as a meningitis. It can also affect other parts of the body including skin, where it may appear as several fluid-filled nodules with dead tissue.

Virtually all cases of cryptococcosis are caused by Cryptococcus neoformans and the closely related species, C. gattii. The global burden of cryptococcal meningitis has been estimated at 223,100 incident cases per year, with 181,100 deaths. The vast majority of patients with cryptococcosis have qualitative or quantitative defects in CD4⁺ T cell function. Cryptococcal meningitis accounts for about 15% of AIDS-related deaths. Other immunosuppressed persons are also at high risk; e.g., solid organ transplant recipients have about 1-5% lifetime risk of developing cryptococcosis. In mouse models of infection, CD4⁺ T cells are also critical for protection, although other arms of the immune system may contribute. Given the public health significance of cryptococcosis, vaccines to protect high risk individuals are a high priority.

### SUMMARY

Provided herein are vaccines for cryptococcosis, including: 1) an antigenic peptide or an antigenic protein and 2) an adjuvant, wherein the antigenic peptide or antigenic protein is derived from a protein listed in Table 1.

In some embodiments, the vaccine includes an antigenic peptide, and the antigenic peptide includes a sequence that is at least 80% identical to an amino acid sequence of any one of SEQ ID NOs: 1-11. In some embodiments, the antigenic peptide has an amino acid sequence of SEQ ID NO: 1. In some embodiments, the antigenic peptide has an amino acid sequence of SEQ ID NO: 9. In some embodiments, the antigenic peptide has an amino acid sequence of SEQ ID NO: 10. In some embodiments, the antigenic peptide has an amino acid sequence of SEQ ID NO: 11. In some embodiments, wherein the antigenic peptide includes a sequence that is at least 80% identical to an amino acid sequence of any one of SEQ ID NOs: 1-11, the adjuvant is either glucan particles or CAF01.

In some embodiments, the vaccine includes an antigenic peptide, and the antigenic protein has an amino acid sequence that is at least 80% identical to an amino acid sequence of any one of SEQ ID NOs: 12-33.

In some embodiments, the antigenic protein has an amino acid sequence that is at least 80% identical to an amino acid sequence of any one of SEQ ID NOs: 12-21. In some embodiments, wherein the antigenic protein has an amino acid sequence that is at least 80% identical to an amino acid sequence of any one of SEQ ID NOs: 12-21, the adjuvant is CAF01.

In some embodiments, the antigenic protein has an amino acid sequence that is at least 80% identical to an amino acid sequence of any one of SEQ ID NOs: 22-33. In some embodiments, wherein the antigenic protein has an amino acid sequence that is at least 80% identical to an amino acid sequence of any one of SEQ ID NOs: 22-33, the adjuvant is glucan particles or CAF01.

Also provided herein are methods of protecting a subject against cryptococcosis infection, the method including: administering to a subject in need thereof any of the above-mentioned vaccines for protection against, or treatment of cryptococcosis infection in the subject.

Also provided herein are methods of protecting a subject against cryptococcosis infection, the method including: 1) generating an antibody using a composition comprising a) an antigenic protein or antigenic peptide derived from a protein listed in Table 1 and b) an adjuvant and 2) administering the generated antibody to the subject in need thereof for protection against, or treatment of cryptococcosis infection in the subject. In some embodiments, the antigenic peptide has an amino acid sequence of any one of SEQ ID NOs: 1-11. In some embodiments, the antigenic protein has an amino acid sequence of any one of SEQ ID NOs: 12-33.

In any of the methods described above, the subject is a human or a koala.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a survival curve showing protection with the GP-Cda2 protein vaccine as a function of inbred mouse strain.

FIG. 2A is a schematic showing the sequences of amino acids 203-232 of the wild-type (Cda2-WT) and the Cda2-M1 and Cda2-M2 mutants.

FIG. 2B is an immunoinformatic analysis showing predicted binding of sequential 15 amino acid peptides spanning amino acids 203-232 of WT-Cda2, Cda2-M1 and M2.

FIG. 2C are survival curves in mice vaccinated with E. coli-expressed Cda2-WT, Cda2-M1, and Cda2-M2 protein loaded into GPs. Control mice received GPs containing mouse serum albumin (MSA). The number of mice in each group: Cda2-Pep1, n=10; Cda2-M1, n=10; Cda2-M2, n=10; MSA, n=5.

FIG. 2D are survival curves similar to FIG. 2C except synthesized 32 amino acid peptides were loaded into GPs rather than E. coli-expressed protein. The number of mice in each group: Cda2-Pep1, n=10; Cda2-M1, n=10; Cda2-M2, n=10; MSA, n=5.

FIG. 3A is a schematic of the sequences of the 32 amino acid peptides, Cda1-Pep1, Cda2-Pep1, Cda3-Pep1, and Fpd1-Pep1.

FIG. 3B are survival curves in BALB/c mice vaccinated with Cda1-Pep1, Cda2-Pep1, Cda3-Pep1, and Fpd1-Pep1 loaded into GPs and then challenged with C. neoformans.

FIG. 4A shows immunoinformatic analysis showing predicted binding to the MHC II IAd allele (present in BALB/c mice) of each sequential 15 amino acid peptide in Cda2 based on the index (start) position of the peptide.

FIG. 4B is a graph showing BALB/c mice received a prime and two biweekly boosts with the indicated peptide encased in GPs and then challenged with C. neoformans, as described in Methods of Example 1.

FIG. 4C shows immunoinformatic analysis, as in FIG. 4A, expect the MHC II IAb allele (present in C57BL/6 mice) was interrogated.

FIG. 4D is graph, as in FIG. 4B except C57BL/6 mice were studied. P=0.0005 for Cda2-Pep5.

FIG. 5 shows the survival curve/protection of BALB/c mice vaccinated with CAF01 adjuvanted recombinant Cryptococcus protein.

FIG. 6 is a graph showing the number of survival days when mice received a titrated amount of Cda1 adjuvanted with CAF01.

FIG. 7 is a survival curve when BALB/c, C57BL/6, and CARD9-deficient mice were immunized on one side of the abdomen with recombinant Cda1 (10 μg) in CAF01 and on the other side with recombinant Cda2 (10 μg) in CAF01.

FIG. 8 are bar graphs of ex vivo stimulation assays of Cda2-CAF01 and Cda1-CAF01 vaccinated mice.

FIG. 9 is a graph shows CFU in the lungs of vaccinated mice following rechallenge with C. neoformans.

FIGS. 10A-10D are bar graphs showing activated CD4 T cells in the lungs of vaccinated mice following rechallenge with C. neoformans. BALB/c and C57BL/6 mice were vaccinated with Cda1-CAF01 and Cda2-CAF01.

FIG. 11 are bar graphs showing interferon gamma production from lung cells of vaccinated and twice challenged mice stimulated ex vivo.

FIGS. 12A and 12B are survival curves showing the contribution of CD4 and CD8 T cells to protection by GP-Cda2.

FIG. 13 is a survival curve showing the effect of depletion of CD4 cells on GP-Cda2 protection.

FIG. 14 is a survival curve showing the effect of partial CD4+ T cell depletion on vaccine-mediated protection.

FIGS. 15A-15D are survival curves showing the contribution of B cells to protection by GP-vaccines.

FIG. 16 are immunofluorescent staining images when C. neoformans was incubated with serum from mice immunized with Cpd1, Lhc1, or Blp4, but not from serum of unimmunized mice.

FIG. 17 is a graphical representation showing the stimulation index of human Th cells by candidate Ags.

### DETAILED DESCRIPTION

Protection against experimental cryptococcosis can be obtained by immunization with cryptococcal strains missing virulence factors such as capsule, chitosan, sterylglucosidase, and F-box protein, or genetically engineered to express interferon-γ. Whole organism vaccines are relatively easy to manufacture and contain a broad range of antigens. However, they may have difficulty reaching clinical trials due to concerns regarding reactogenicity, autoimmunity and, if administered live, the possibility of causing infection in immunosuppressed persons. Subunit vaccines may help to circumvent these potential drawbacks.

### General Definitions

As used herein, the articles “a” and “an” refer to one or to more than one (e.g., to at least one) of the grammatical object of the article.

The term “or” is used herein to mean, and is used interchangeably with, the term “and/or,” unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies, the term “polypeptide” encompasses polypeptides as a single chain and polypeptides of two or more associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid identity, when compared and aligned for maximum correspondence, as measured using a sequence comparison algorithm or by visual inspection. In some embodiments, identity exists over a region of the sequences that is at least about 10, at least about 20, at least about 20-40, at least about 40-60, at least about 60-80 nucleotides or amino acids in length, or any integral value there between. In some embodiments, identity exists over a longer region than 60-80 nucleotides or amino acids, such as at least about 80-100 nucleotides or amino acids, and in some embodiments the sequences are substantially identical over the full length of the sequences being compared, for example, (i) the coding region of a nucleotide sequence or (ii) an amino acid sequence.

The phrase “conservative amino acid substitution” as used herein refers to a substitution in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of an alanine for a valine is considered to be a conservative substitution. Methods of identifying nucleotide and amino acid conservative substitutions that do not eliminate binding are well-known in the art.

The term “vector” as used herein means a construct that is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

The term “isolated” as used herein refers to a polypeptide, soluble protein, antibody, polynucleotide, vector, cell, or composition that is in a form not found in nature. An “isolated” antibody is substantially free of material from the cellular source from which it is derived. In some embodiments, isolated polypeptides, soluble proteins, antibodies, polynucleotides, vectors, cells, or compositions are those that have been purified to a degree that they are no longer in a form in which they are found in nature. In some embodiments, a polypeptide, soluble protein, antibody, polynucleotide, vector, cell, or composition that is isolated is substantially pure. A polypeptide, soluble protein, antibody, polynucleotide, vector, cell, or composition can be isolated from a natural source (e.g., tissue) or from a source such as an engineered cell line.

The term “substantially pure” as used herein refers to material that is at least 50% pure (i.e., free from contaminants), at least 90% pure, at least 95% pure, at least 98% pure, or at least 99% pure.

The term “subject” refers to any animal (e.g., a mammal), including, but not limited to, humans, non-human primates, canines, felines, rabbits, rodents, koalas and the like.

The term “pharmaceutically acceptable” as used herein refers to a substance approved or approvable by a regulatory agency or listed in the U.S. Pharmacopeia, European Pharmacopeia, or other generally recognized pharmacopeia for use in animals, including humans.

The terms “pharmaceutically acceptable excipient, carrier, or adjuvant” or “acceptable pharmaceutical carrier” as used herein refer to an excipient, carrier, or adjuvant that can be administered to a subject, together with at least one therapeutic agent, and that is generally safe, non-toxic, and has no effect on the pharmacological activity of the therapeutic agent. In general, those of skill in the art and government agencies consider a pharmaceutically acceptable excipient, carrier, or adjuvant to be an inactive ingredient of any formulation or any pharmaceutical composition.

The term “pharmaceutical formulation” or “pharmaceutical composition” as used herein refers to a preparation that is in such form as to permit the biological activity of the agent to be effective. A pharmaceutical formulation or composition generally comprises additional components, such as a pharmaceutically acceptable excipient, carrier, adjuvant, buffers, etc.

The term “effective amount” or “therapeutically effective amount” as used herein refers to the amount of an agent that is sufficient to reduce and/or ameliorate the severity and/or duration of (i) a disease, disorder or condition in a subject, and/or (ii) a symptom in a subject. The term also encompasses an amount of an agent necessary for the (i) reduction or amelioration of the advancement or progression of a given disease, disorder, or condition, (ii) reduction or amelioration of the recurrence, development, or onset of a given disease, disorder, or condition, and/or (iii) the improvement or enhancement of the prophylactic or therapeutic effect(s) of another agent or therapy (e.g., an agent other than the binding agents provided herein).

The term “therapeutic effect” as used herein refers to the effect and/or ability of an agent to reduce and/or ameliorate the severity and/or duration of (i) a disease, disorder, or condition in a subject, and/or (ii) a symptom in a subject. The term also encompasses the ability of an agent to (i) reduce or ameliorate the advancement or progression of a given disease, disorder, or condition, (ii) reduce or ameliorate the recurrence, development, or onset of a given disease, disorder, or condition, and/or (iii) to improve or enhance the prophylactic or therapeutic effect(s) of another agent or therapy (e.g., an agent other than the binding agents provided herein).

The term “treat” or “treatment” or “treating” or “to treat” or “alleviate” or alleviation” or “alleviating” or “to alleviate” as used herein refers to both (i) therapeutic measures that aim to cure, slow down, lessen symptoms of, and/or halt progression of a pathologic condition or disorder and (ii) prophylactic or preventative measures that aim to prevent or slow the development of a targeted pathologic condition or disorder. Thus, those in need of treatment include those already with the disorder, those at risk of having/developing the disorder, and those in whom the disorder is to be prevented.

The term “prevent” or “prevention” or “preventing” as used herein refers to the partial or total inhibition of the development, recurrence, onset, or spread of a disease, disorder, or condition, or a symptom thereof in a subject.

### Cryptococcal Vaccines

Provided herein are subunit vaccines for the protection against Cryptococcus. Instead of the entire pathogen, “subunit vaccines” include only the components, or antigens, that best stimulate the immune system.

### Antigenic Proteins or Antigenic Peptides

A vaccine capable of protecting at-risk persons against infections due to Cryptococcus neoformans and Cryptococcus gattii could reduce the substantial global burden of human cryptococcosis. Vaccine development has been hampered by lack of knowledge as to which antigens are immunoprotective and the need for an effective vaccine delivery system. As shown herein, immunoprotective antigens of Cryptococcus were identified by extracting antigens from Cryptococcus by treatment with an alkaline solution or by expressing Cryptococcus proteins recombinantly in E. coli. The term “antigen” refers to one or more epitopes that can elicit an immune response.

The compositions described herein feature an antigen conjugated, mixed, or associated with an adjuvant. In some instances, the antigen is an antigenic protein. Preferred antigenic proteins include long, synthetic/recombinant molecules that are made up of 50 or more amino acids. In other instances, the antigen is an antigenic peptide. Preferred antigenic peptides include short, synthetic/recombinant molecules that are between 5 and 50 amino acids in length, for example, 5-10, 5-15, 5-20, 5-25, 5-30, 5-35, 5-40, 5-45, 10-15, 10-20, 10-25, 10-30, 10-35, 10-40, 10-45, 15-20, 15-25, 15-30, 15-35, 15-40, 15-45, 20-25, 20-30, 25-35, 25-40, 25-45, 30-35, 30-40, 30-45, 35-40, 35-45, 40-45, or 45-50 amino acids in length.

A list of proteins from which the antigen (either protein, i.e., molecule of 50 or amino acids, or peptide, i.e., less than 50 amino acids) may be derived from are included in Table 1 below. See also, Hester M M, Lee C K, Abraham A, Khoshkenar P, Ostroff G R, Levitz S M, Specht C A. Protection of mice against experimental cryptococcosis using glucan particle-based vaccines containing novel recombinant antigens. Vaccine. 2020 Jan. 16; 38(3):620-626. doi: 10.1016/j.vaccine.2019.10.051. Epub 2019 Nov. 4.; broadinstitute.org/fungal-genome-initiative/Cryptococcus-neoformans-serotype-genome-project; GenBank RefSeq Assembly GCA_000149245.3; and ncbi.nlm.nih.gov/bioproject?LinkName=nuccore_bioproject&from_uid=405117359; all of which are incorporated herein by reference.

TABLE 1

| Name | CNAG_xxxxx | C. gattii ortholog | Description |
|---|---|---|---|
| Cda1 | 05799 | CNBG_1745 | chitin deacetylase |
| Cda2 | 01230 | CNBG_9064 | chitin deacetylase |
| Cda3 | 01239 | CNBG_0806 | chitin deacetylase |
| App1 | 06574 | CNBG_1982 | antiphagocytic protein |
| Lhc1 | 04753 | CNBG_6019 | lactonohydrolase |
| Fpd1 | 06291 | CNBG_5149 | deacetylase |
| Sod1 | 01019 | CNBG_0599 | superoxide dismutase |
| MP88 | 00776 | CNBG_1155 | mannoprotein |
| Cpd1 | 00919 | CNBG_6045 | carboxypeptidase |
| YjeF | 05097 | CNBG_4593 | ribokinase |
| PTP | 07442 | CNBG_0642 | phosphotidylinositol |
| | | | transfer protein |
| DHA1 | 07422 | CNBG_0934 | delayed-type |
| | | | hypersensitivity protein |
| Nuc | 00264 | CNBG_0307 | nuclease/phospholipase |
| Glo1 | 02030 | CNBG_5182 | glyoxal oxidase |
| 3223 | 03223 | CNBG_2366 | unknown |
| ACK | 06432 | CNBG_5028 | acetate kinase |
| Blp4 | 01562 | CNBG_3874 | unknown |
| Rds1 | 06267 | CNBG_9636 | ferritin-like |
| Sacch | 00581 | CNBG_1355 | saccharopepsin, |
| | | | aspartic protease |
| Cerev | 04625 | CNBG_1027 | cerevisin, peptidase |
| Mep1 | 04735 | CNBG_6001 | metallo proteinase |
| 3492 | 03492 | CNBG_2122 | unknown |
| May 1 | 05872 | CNBG_1672 | aspartic protease |
| 3143 | 03143 | CNBG_2441 | heat shock protein 9/12 |
| 4874 | 04874 | CNBG_5802 | glucanase |
| Kpr1 | 05595 | CNBG_4278 | unknown |

### Exemplary Antigenic Peptides

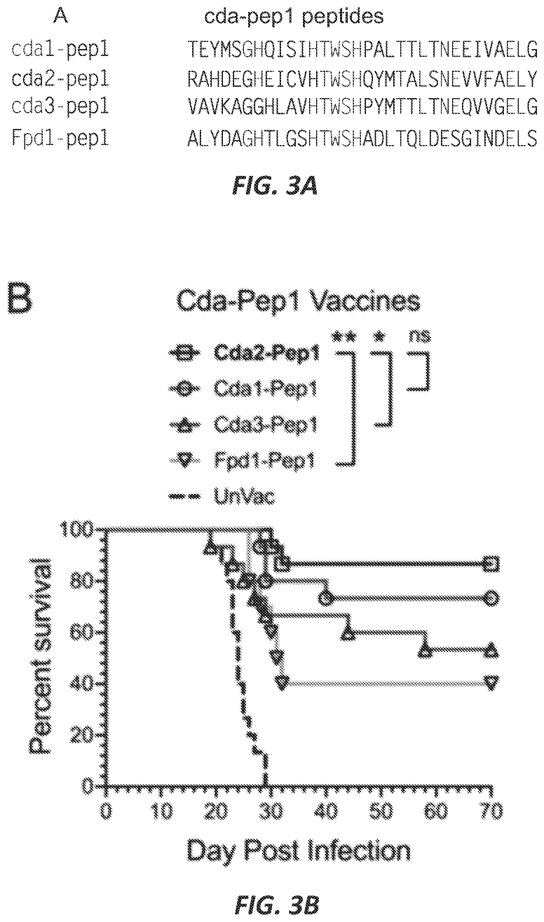

Exemplary peptides are shown in Tables 2 below and FIG. 3A (Cda1-Pep11, Cda2-Pep1, Cda3-Pep1, SEQ ID NOS: 9, 1, 10, 11, respectively).

TABLE 2

| | | Amino | Index | SEQ |
|---|---|---|---|---|
| | | Acid | Posi- | ID |
| Peptide | Sequence | Position | tion | NO: |
| Cda2- | RAHDEGHEICVHTWSH | 203-234 | 203-220 | 1 |
| Pep1 | QYMTALSNEVVFAELY | | | |
| Cda2- | ENNQKATMFFIGSNVL | 180-210 | 180-196 | 2 |
| Pep2 | DWPLQAMRAHDEGHE | | | |
| Cda2- | TWGLGFDDGPNCSHNA | 158-189 | 158-175 | 3 |
| Pep3 | LYDLLLENNQKATMFF | | | |
| Cda2- | EVVFAELYYTQKAIKAV | 227-260 | 227-246 | 4 |
| Pep4 | LGVTPQCWRPPYGDVDN | | | |
| Cda2- | TAYSYAPVTELISSFPT | 60-91 | 60-77 | 5 |
| Pep5 | IWQTASIPSNDTEAQ | | | |
| Cda2- | MSVFMTMFPKIKSAFNY | 328-361 | 328-347 | 6 |
| Pep6 | IVPICTAYNITQPYAES | | | |
| Cda2- | DKAGNGTYTTHGPVVLNH | 304-338 | 304-324 | 7 |
| Pep7 | ELTNYTMSVFMTMFPKI | | | |
| Cda2- | KINSTLNTKIPNDVPHGT | 96-130 | 96-116 | 8 |
| Pep8 | PTGDWTGVNYSNSDPDC | | | |

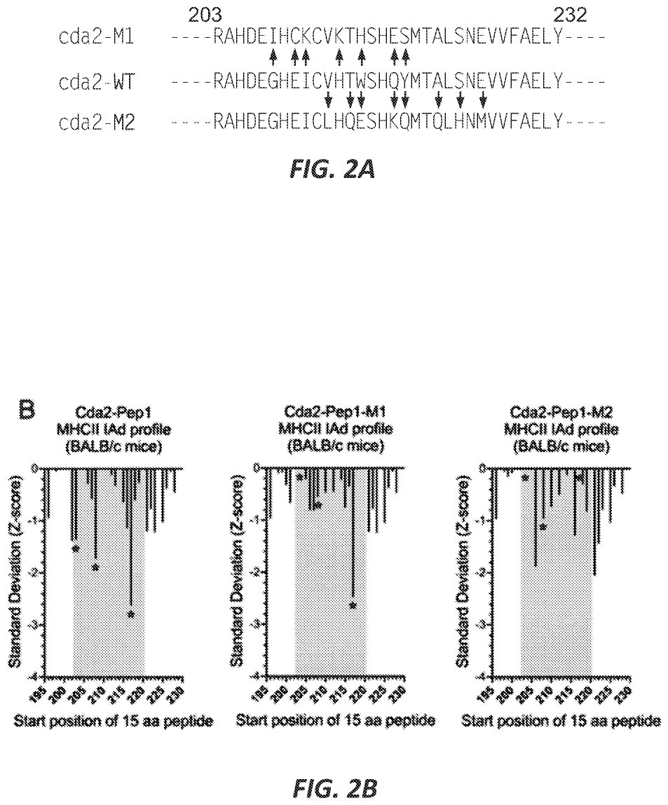

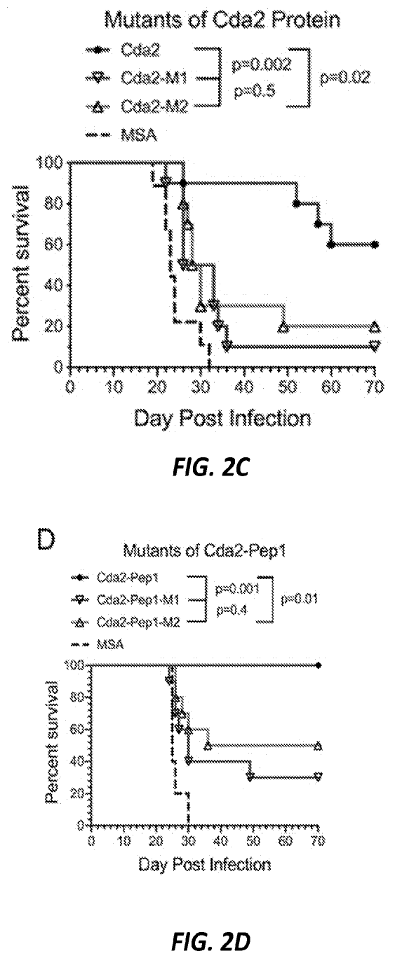

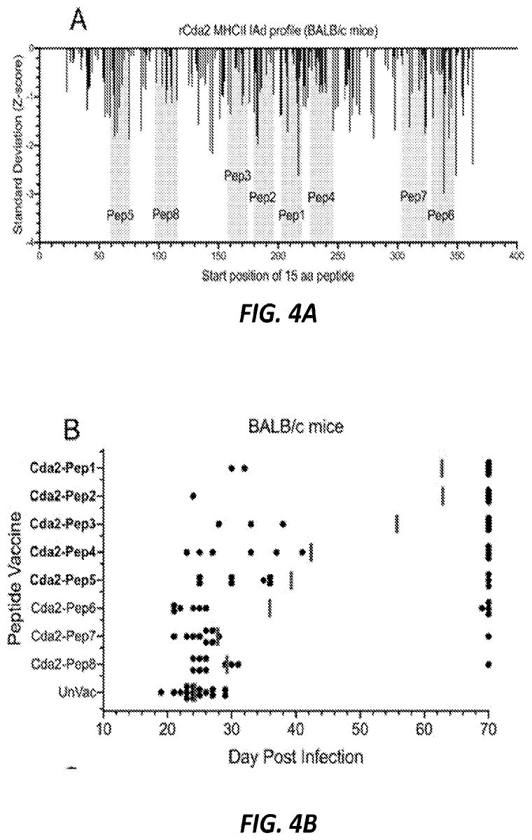

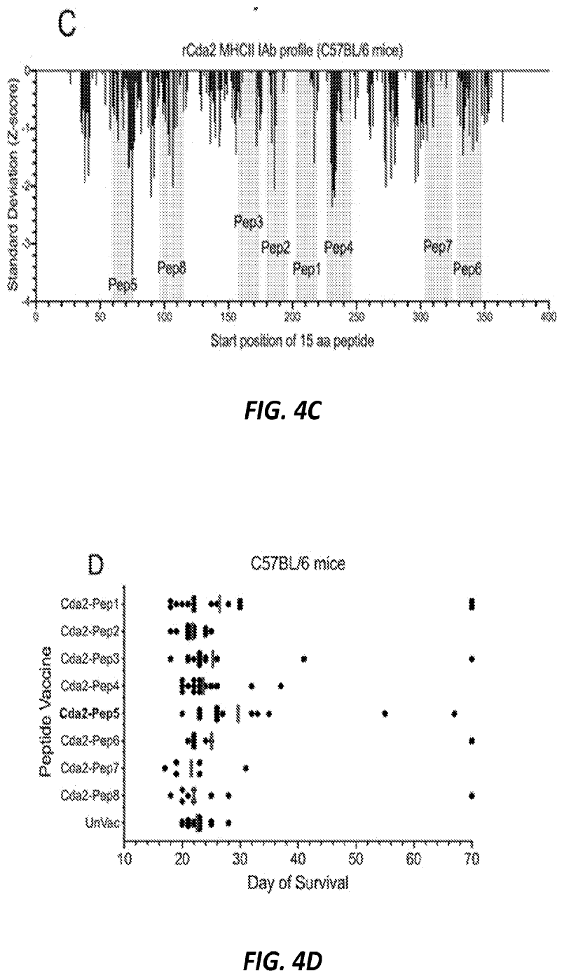

Table 2 shows the sequences of Cda2 peptides used in vaccines shown in FIG. 4. Protein sequence of Cda2 that was used is from GenBank Accession XP_012049402.1 (see also SEQ ID NO: 14, below). The index (start) position refers to the first amino acid in a 15 amino acid peptide. The index position is used to identify the vertical bars that depict relative binding of the peptide to a MHCII allele shown in FIGS. 2B, 4A, and 4C. Thus, in FIG. 2B, the vertical bar for Cda2-Pep1 at amino acid 203 refers to the peptide sequence spanning amino acids 203-217.

### Protein Sequences

“Percent (%) amino acid sequence identity”, “Percent (%) sequence identity” or “Percent (%) identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In some of the embodiments provided herein, the antigenic protein or antigenic peptide may have at least 60%, at least 65%, at least 70%, at least 75%, at least 80%, at least 85%, at least 90%, at least 95%, at least 96%, at least 97%, at least 98%, at least 99%, or 100% identity to any of the sequences listed above in Table 2 (i.e., SEQ ID NOs:1-8, FIG. 3A (i.e., SEQ ID NOs: 9-11), or any of the sequences listed below (i.e., SEQ ID NOs: 12-33).

Exemplary sequences are listed below.

Cda1 (CNAG_05799)
(SEQ ID NO: 12)
mftfaafsal lislagvvaq ttgtsvdssi ltktadstgp sgfsipalse ltsgaptdst
valystfaag atptvsgapv lptsaltiad ypaldvtppt nsslvkdwma kidlskvpsy
nvttgdcstd aaaisdgrcw wtcggctret divecpdknv wglsyddgps pftpllidyl
qeknikttff vvgsrvlsrp emlqteymsg hqisihtwsh palttltnee ivaelgwtmk
vikdtlgvtp ntfrppygdi ddrvraiaaq mgltpviwts ytdgsttvnf dtndwhisgg
tatgassyet fekilteyap kldtgfitle hdiyqqsvdl avgyilpqvl angtyqlksi
inclgkdtse ayietssnqt ttqitaatgs qstffqpivg tatgaevsap seatgstaag
saasttsgsg asastgaasn tsssgsgrsa tmggaliala avavgmvyva

Cda1 (CNBG_1745) is incorrectly annotated in the publicly available genome database; the re-annotated sequence is provided below. The reannotated version includes an exon that results in a sequence of similar length and greater homology to CNAG_05799.

Cda1 (CNBG_1745)
(SEQ ID NO: 13)
msafaavsal lvslagimaq tastsvassv ltqtaptgps gfsipalsel tsgaptdttv
alystfpasa tptvsgapvl ptsaltiany paldvipptn sslvqewlak idmtkvpnyn
attgdcstdp gatsdgrcww tcggctratd ivacpdknvw glsyddgpsp ftpllidylq
eknikttffv vgsrvlsrpe mlqteymsgh eisihtwshp alttltneqi vaelawtmkv
ikdtigvtpn tfrppygdid drvraiaaqm gltpviwtsy sdgsttvnfd tndwhisggt
atgassyetf ekilneyapk Intgfitleh dlyqqsvdla vgyilpqvla ngtyqlksii
nclgkdisea yietssnqtt tqitsasgst yfqpivgtat gsevsapsea tgstaagsaa
gsaattsgsa asgasdssss gsgrsatmgg aliafaavav givyva
Cda2 (CNAG_01230)
(SEQ ID NO: 14)
mipstaaall tltagaafah tgcggheigr rnvggpmlyr ravtdeasaa vstdintect
aysyapvtel issfptiwqt asipsndtea qqlfgkinst Intkipndvp hgtptgdwtg
vnysnsdpdc wwthnkcttp sndtglqadi siapepmtwg lgfddgpncs hnalydllle
nnqkatmffi gsnvldwplq amrahdeghe icvhtwshqy mtalsnevvf aelyytqkai
kavlgvtpqc wrppygdvdn rvrmiaegln Ittiiwsddt ddwaagtngv teqdvtnnyq
svidkagngt ytthgpvvln heltnytmsv fmtmfpkiks afnyivpict aynitqpyae
snitcpnfet yisgvtniss sttqkdgsss tntasgsgaa gsasatsssd dssssggssg
ssgsnnassg algmfdslsg vglilggvva gvmll
Cda2 (CNBG_9064)
(SEQ ID NO: 15)
mipstaaafl tltagtafah igcggqeigr rnvggpmlhs ravtdeasaa vstdvstect
aygyapvtqi assfpaiwqt asilstdsea qqlfasinat vnsklpndvp hgtptgnwtg
vsysssdpdc wwthnkcttp ssdtglkadi ttvpepmtwg lgfddgpncs hnalydllle
nnqkatmfyi gsnvmdwplq amrahdeghe icvhtwshqy mtalsnevvf aelyytqkai
kavlgvtplc wrppygdvdn rvrmiaaaln lstivwsddt ndweagtngv tqqdvtnnyq
svidkagngt ytthgpvvln heltnytmsv fvsmfpkiks afsyivpict aynitqpyae
snvtcpnfet yisgvtnist sttqkdgsss tntsytasgs tspsasstgk sddssssgsa
ssstaansas saksgalgmy dglsgmglil ggvvagvmll
Cda3 (CNAG_01239)
(SEQ ID NO: 16)
myghlslsal slfavvaaap freswlqprd spvsqlfrrt apdpnsndym syypgpgstp
nvstipqawl dklatvnlpn vpvatpdggr ptypnneddg dsticsftdq crveddlysp
pgekiwalsf ddgptdvspa lydylaqnni sssathfmig gnvitspqsv lvavkagghl
avhtwshpym ttltneqvvg elgwtmqals dinggripmy wrppygdvdn rvraiakevf
glvtvlwdsd tndwaitdep gqysvasvea yfdtlvtgnr tqgllllehe ldnntvevfe
teypkavgng wtvknvadaf nmewylnsgk gnndvvttms vagtlttatp tntstyvass
taassasvtd sagvsiasaa sseassswai anrpshfvia iacglalaai mv
Cda3 (CNBG_0806)
(SEQ ID NO: 17)
myghlslsal sllavvaaap fheswlqprd spvsqlfrra apdpnasdyl shypspgstp
nvstipqawl dklatvqlpn vsvatasgei ptypnnendg dsticsftdq cvepddlfsp
pgekiwalsf ddgptdvspg lydflaqnni sskathfmig gnvvtspqsv liavqagghl
avhtwshpym ttltneqvvg elgwtmqals dlnggrvpkf wrppygdvdn rvraiakgvf
dletvlwded tndwaiadep sqysiasvea yfdtlvtgnr tqgllllehe ldnntvtvfe
teypkaiang wivknvadaf nmewylnsgk gndatvttms vggtlptaap tntstsvasa
satssgsvtd sagvsiasaa ssesssswai aerpslfiia cglvfaaavv
Fpd1 (CNAG_06291)
(SEQ ID NO: 18)
mkfittlfav lailssvsas ptmkkratve tidncsqqgt valtfddgpy dyeaqvasal
dggkgtffln ganyvciydk adsiralyda ghtlgshtws hadltqldes gindelskve
dafvkilgvk pryfrppygn indnvlkvlg ergytkvflw sddtgdange svsysegvld
gviqdypnph lvldhstiet tssqvlpyav pklksagyql vtvgeclgtd espyewvdcp
gerdsswqc
Fpd1 (CNBG_5149)
(SEQ ID NO: 19)
msldrpyggv pssfalpyri pfairllpfl plskeykcqp sttsllfflq pssdkeksyp
ikmkfittff tvlailssas asptvkkrat vetidncsqq gtvaltfddg pynyeaqvas
aldggkgtff Inganyaciy dkadsikaly daghtlgsht wshadltqld esgindelsk
vedafvkilg vkplyfrppy gnindnvlnv lsqrgykkvf mwsddtgdan gesvsysegv
ldkviqnypn phlvlehspi ettssqvlpy avpklknagy qlvtvgeclg tnespyefvg
cpgerdsswq c
Blp4 (CNAG_01562)
(SEQ ID NO: 20)
mfakaavial asasivaaap vncarakptt ydegylesyd syharylals cysqhnttff
ddcchpllat etladnrasy ctpnstavas vnatiaeata satasadiea esqynnassy
aaeatapvtt saeatapvta saeatasvta aavnnvaeva qqsasassee eqptasssss
ygrasassss seeeststss ssasdsssts ssqvytggya tffsqggvag ecgtvhsddd
yviaidsngw wqdyesndss pycgkhitlt ntnngksvta vvadvcptce tansldlsig
afnqiateed gmvpitwyft d
Blp4 (CNBG_3874)
(SEQ ID NO: 21)
mfakaavial asasivvaap vncarskpst yaegyledyd tyharylals cntqhnttff
ddcchpllan etladnrasy ctpnstavas inatmaegaa atasatasad veaesqynna
sssdatasvt asseataset asalnnvaei aqqsasatss seeqptsass saseesaast
ssssastsss tassqvytgg yatfysqdgv agacgtmhsd sdyviaidsn gwwqdyesnd
nspycgkqit ltntnngksv tatvadvcpt cetnnsldls igafnqiate eegmvpitwy
fvn
App1 (CNAG_06574)
(SEQ ID NO: 22)
mmssataelc fdcancveml pkgttksvtr peilerltri enhievqtal Istmaaskdy
ekkvytdddd eleepevqlc nmtcemaalk tykpqtkvsn hkivieaalg rldhvngess
irwaqsvilg lqaaetlqnd kqmmelarvi gcleacelrw agdwragvas itlkelrtlm
i
App1 (CNBG_1982)
(SEQ ID NO: 23)
mmssapselc fdcancveml pkgtkksatr peilerltri edrieiqttl lrgmatdrdy
ekkvytdddd eleesevpvc nitremaalk tykaqpkvtn hkviieaalg rldhvngdns
irwaqsvilg lqaaetlqnd rommelarvi gcleacelrw agdwragvas itlkelrtlm
i
Lhc1 (CNAG_04753)
(SEQ ID NO: 24)
matnknpdph akitsremsv ntaglqewgm ggktlpaplg spvkfilgak vlswksklvy
agftmgalat laytashilp sssiaqashl ttfkrratps eppknaqvin pkdftvlptv
lpsyefngss lfvppgtteq slkakpfhvy ddsfydvigs nptltliads gsdplfheav
vwykatdevf fvqnagaksa gtglnksavv ekislleaaa vqkgekheaq vtvvnstvqv
vnpnggtayr gkivfagegq ganvppalym ldpnepyptt vilnnyfgrq fnslndisvn
prnkelyftd vtygylqdfr pppglpnqvy rfnmdtgvit vvadglnmpn gltfspdgrh
ayvtdtgifs gfwgtnytyp atiyrwdvqd dgtwenqkvf ayihvgaadg ihtdsngnvy
agvgdgvhvf nphglligki ylgetsanfn fagngrmiic aethlyyatl gasgwdpea
Lhc1 (CNBG_6019)
(SEQ ID NO: 25)
msinsadlqe remegktlpa plgssmksip gakgfswkpk fvymglmaal atlayitiyt
lpfaviarap shlstfkrrs gpreppeyaq riypkefavl dtvppptefd gsslfvppgt
tkeslkakpf hiyddsfldi igtnptltli adsgtnplfh eavvwytvkd evlftqnaga
kaagtglnks aviekillse aaavqkhern qtevfvvnst vqvmnpngat pygdkfifag
qgqgpnvppa lymldpeepy httiilnnff grqfnsindv svnprngeiy ftdvmyaylq
dfrpapglpn qvyrfnletg lvqvvadgin mpngitfspd grhayvsdta ifsafwgtny
typatiyrwd veddgtwsnr klfayihvga adgihtdshg nlyagvgdgi hvfnpsgmli
gkiylgetsa nfnfaghgrm vicaethlyy atlgvsgwvp kaktv
Kpr 1 (CNAG_05595)
(SEQ ID NO: 26)
mfavaalasl lsaiavkavp cvqfdsswnl yafggdqdvm igdnttwssp sttplstsgr
ppwtgnntqc ilsqtnnamy vigadsddls siyvydfsgd swstqstsrt psdlgnsrss
tvldhdtnvf ftltidsgly qldlssirns asrdtlrwea vsnpsfsvdd ytvtaaqaan
hifffgtpgt aagsahifvv hyayfqpeaq afngtafpna agqaisipnv dnnvpysmvf
ipddfsdtyi vthwtdlsny svtsdapfds dlinstqtlp aptsqdataa yaaspyalvq
vdaagdiyym snpvqsdytv sssasweklg ysligvrest snnsssssta sststatgaa
agsasgsvtr ssstgdasas assasnsssg arkmatrgda lglsmgalvl vagilm
Kpr1 (CNBG_4278)
(SEQ ID NO: 27)
mlaaaalasl latiavnavt cvqfdsswnl yafggdqdvk igdnntwssp sttplsttgr
ppwtgnntqc ilsqtnnamy vigadsddls siyvydfagn swstqntsrt psdlgnsrss
svldhdtnvf ftlttdsgly qldlssitns assdtlrwea venpsfsvdg yfvtaagaan
hifyfgapga asgsayifvv hyayfqpkaq afngtafpda sgqaisipsa annvpysmvf
iphdfsdtyi vthwtdlsdy svtsdapfdv nlinstqtlp aptsqdkaaa yaaspyaivq
idaagdiyym sspvqsdytv sssasweklg ysltlskskd tssssttsgt stttgathgs
asgtasrpgs tdnasasast nsssgarrma trgdvlglfv galavaagil m
Cpd1 (CNAG_00919)
SEQ ID NO: 28)
mcskvvsavl lafalgsvie aarephglrg rgpaalaake akiealanna snndaskveq
crtpkerhpk wrfynnktse flikslpdvp fdlgeiysgl ipidyhnqse glffvfqpkl
geasdditiw Inggpgcssl egwfqenglw twqagtyapv inhyswvnlt nmlwveqpig
tgfsigtpka tteeeiaqdf ikwfknfqdl fgiknykiyv tgesyagryv pyigaamlda
qnktyydlsg alvydpaige svfvqeqitt ypfveananl fnfnkttmae fkelhetcgy
kdyierylkf pptenqpplf fdyydidnvt caifdwvike afrinpcfdi yeinlmcpll
wdvlgmptqf syapggiyfn rsdvkaaiha pehidwtlca trpvfvggee qgpqgrgdls
adpiqkvlpq vieatnrvli sngdfdmvii tngtllaiqn mtwnghlgfq sppsedifid
ivdtqwssif esngyhgypg aqgvmgiqhy erglmwaqty qsghmqpryq prssyrhlqw
llgyvdrl
Cpd1 (CNBG_6045)
SEQ ID NO: 29)
mwskvvtval laialgsiie ardfhnlrgr gspalatnke akkevlttrq sktkdkchar
percpemkrf yneqtseffv eslpdvpfdl geiysglipi dygnqsealf fvfqpklgec
sddltiwlng gpgcssligf fqenglwtwq pgtyapvinp yswvnltnml wveqpigtgf
sigtpkatte eeiaqdfikw fknfqdifgi knykiyvsge syagryvpyi gaamldqqdk
tyydlsgalv ydpaigetif vqeqiptypf veananlfnf dkttmaelke lhetcgyqdy
idrylkfppt enqphlyydy fdydnatcgi fdkvlakasr inpcfdiyai nqmcpllwdi
lgtptqldya pggiyfnrsd vkasihapsh idwtacatqp vfvggedgpq srgdvsldpi
qkvlpqviea tnrvlisngd fdyviltngt llsiqnmtwn gqlgfqsapn edividiidt
qwssvyeany regypgaqgv mgiqhyergl mwaetfqsgh mqpqfqprsa yrhlqwllgh
idkl
3143 (CNAG_03143)
SEQ ID NO: 30)
msdagrqsft dkagaamkpd seksyleqak dtiggkadsa astgqpqsqk sytqeigdaf
sgnkndnges ltdkaknafg anq
3143 (CNBG_2441)
SEQ ID NO: 31)
msdtgrqslt dkagaalkpd seksyleqak dtisgktdsa astaqpqsqk sytqeigdaf
sgnkndnqes ltdkaknafg anq
4874 (CNAG_04874)
SEQ ID NO: 32)
mltllaialp itialragat vyplveswhg egffdgftfp vetydnttng dtfwatpant
sllyttssgt tilkvdnstf vpylekrfap kllsksaydi gtvwvfdavh lpygcsvwpa
fwtqgpswpa ggeidivegi nlqatnmial htsgasscti pttspssfsg tvsypncdns
qnygsgctvy dtntnsygre faeagggvyv aefardgiri wfmtrsaipd aiqvnatqid
tsslgtpvae ypstscdias lfgpqtltin ialcgdyagl pselektcpa lvgdatcytt
yvindgstty sqayfeinyv nvyssnpssv ttispsgpst satsttasts aagtreggat
rpeqqlllva agslmslflf w
4874 (CNBG_5802)
SEQ ID NO: 33)
mmflsdkpnp icvimdgkdq dattdrgslr fvffrdtasp cligrqriml lssqkdgyik
crswtdpInt nlstdvdltp nrlanypsas swgdslpldr vlardgfrfp petydnttng
dtfwetaant sllyttsagt tilkvdnttf vpylekryap kllsksaydl gtvwvmdavh
mpygcsvwpa fwtqgpswpa ggeidiiegi nlqptnmial htsgntscti pttftpsfsg
rvsypncdns qnfgsgctvy dpnpnsygqa faeagggvfv aefaedgirv wfmtrsaips
tvrvnatqid tstlgipvae ypssscditn lfgpqtltin ialcgdyagl pselartcpa
lvgdatcytt yvinnastty agayfeinyi nvyssnpstf ttispsgptp tsstststst
sastpdagtl sggarrvere svlvamgllv nffhfgsilf gge

### Adjuvants

As used herein, the term “adjuvant” refers to an agent that increases the immune response to an antigen (e.g., cryptococcal surface antigens). As used herein, the term “immune response” refers to a subject's (e.g., a human or another animal) response by the immune system to immunogens (i.e., antigens) the subject's immune system recognizes as foreign. Immune responses include both cell-mediated immune responses (responses mediated by antigen-specific T cells and non-specific cells of the immune system—Th1, Th2, Th17) and humoral immune responses (responses mediated by antibodies). The term “immune response” encompasses both the initial “innate immune responses” to an immunogen (e.g., cryptococcal surface antigens) as well as memory responses that are a result of “acquired immunity.”

### Glucan Particles

In some instances the subunit vaccine may be adjuvanted to a glucan particle. Glucan particles (GPs) are composed primarily of β-1,3-glucan and are devoid of proteins, lipids, and mannans. GPs are recognized by the C-type lectin receptor Dectin-1, but also potently activate the alternative pathway of complement. In vivo, phagocytosis of GPs is mediated by both complement receptors and Dectin-1. GPs stimulate dendritic cells (DCs) to produce cytokines associated with beneficial responses in vaccine models of protection.

Briefly, the process for producing the glucan particles (GPs) involves the extraction and purification of the alkali-insoluble glucan particles from the yeast or fungal cell walls. The structure-function properties of the glucan particle preparation depend directly on the source from which it is obtained and also from the purity of the final product. The source of glucan particles can be yeast or other fungi. The yeast strains employed in the present process can be any strain of yeast, including, for example, S. cerevisiae, S. delbrueckii, S. rosei, S. microellipsodes, S. carlsbergensis, S. bisporus, S. fennentati, S. rouxii, Schizosaccharomyces pombe, Kluyveromyces polysporus, Candida albicans, C. cloacae, C. tropicalis, C. utilis, Hansenula wingei, H. ami, H. henricii, H. americana, H. canadiensis, H. capsulata, H. polymorpha, Pichia kluyveri, P pastoris, P polymorpha, P rhodanensis, P ohmeri, Torulopsis bovin, and T. glabrata. Alternatively, mutant yeast strains can be employed.

### CAF01

CAF01 is an adjuvant that has an acceptable safety profile and proved successful in preclinical development of antibody and Th cell activating vaccines against malaria, chlamydia, and tuberculosis, and are currently in clinical trials. CAF01 induces very robust memory T cell responses. CAF01 consists of dimethyldioctadecyl-ammonium (DDA) and trehalose 6,6′-dibehenate (TDB). DDA is a synthetic amphiphilic lipid compound comprising a hydrophilic positively charged dimethylammonium head-group attached to two hydrophobic 18-carbon alkyl chains (tail). In an aqueous environment, DDA self-assembles into closed vesicular bilayers (liposomes). The adjuvant efficacy and stability of the liposomes (DDA) are increased by incorporation of the synthetic glycolipid TDB which is a synthetic analogue to the immune stimulatory component of the mycobacterial cell wall often referred to as the cord factor or trehalose dimycolate.

### Conjugation/Linker/Encapsulation

In exemplary aspects the antigenic protein or antigenic peptide is trapped inside the glucan particle thereby allowing the antigenic protein or antigenic peptide to be encased by the adjuvant. The antigenic protein or antigenic peptide may also be admixed with adjuvant, such as CAF01, to create a vaccine formulation.

### Pharmaceutical Compositions

According to a further aspect, the adjuvanted antigenic proteins or adjuvanted antigenic peptides are provided herein for use in medicine. In other words, the adjuvanted antigen containing peptide are provided for use as a medicament.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Pharmaceutical compositions are typically formulated to be compatible with its intended route of administration, the present methods will typically include local intramuscular injection thus formulation for parenteral administration is desirable.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

The pharmaceutical compositions can be included in a container, pack, or dispenser together with instructions for administration.

### Methods of Administration

The methods described herein include methods for the diagnosis, prevention and treatment of disorders associated with cryptococcosis. In some embodiments, the disorder is anything associated with cryptococcosis (e.g., chest pain, dry cough, headache, nausea, confusion, stupor, coma, neck stiffness, blurred or double vision, fatigue, fever, unusual and excessive sweating at night, swollen glands without the appearance of infection in nearby areas, skin rash, pinpoint red spots (petechiae), bleeding into the skin, bruises, unintentional weight loss, appetite loss, abdominal bloating, abdominal pain, abdominal swelling, weakness, bone pain, and numbness and/or tingling). Generally, the methods include administering a therapeutically effective amount of an adjuvanted antigen containing peptide as described herein, to a subject who is in need of, or who has been determined to be in need of, such treatment.

As used in this context, to “treat” means to ameliorate and/or prevent at least one symptom of the disorder associated with cryptococcosis. Often, cryptococcosis results in any of the symptoms mentioned above (i.e., chest pain, dry cough, headache, nausea, confusion, stupor, coma, blurred or double vision, neck stiffness, blurred or double vision, fatigue, fever, unusual and excessive sweating at night, swollen glands without the appearance of infection in nearby areas, skin rash, pinpoint red spots (petechiae), bleeding into the skin, bruises, unintentional weight loss, appetite loss, abdominal bloating, abdominal pain, abdominal swelling, weakness, bone pain, and numbness and/or tingling); thus, a treatment can result in a reduction and/or prevention in any of those symptoms. Administration of a therapeutically effective amount of a compound described herein for the treatment of a condition associated with Cryptococcus will result in a prevention or decrease of the above mentioned symptoms.

### Kits

According to further embodiments, kits are provided comprising an adjuvanted antigen containing peptide and a pharmaceutically acceptable excipient.

Alternatively, also provided herein are kits comprising an antigen containing peptide, an adjuvant, a pharmaceutically acceptable excipient, and instructions for preparing an adjuvanted antigen containing peptide suitable for administration to a patient/subject in need thereof.

### EXAMPLES

The invention is further described in the following examples, which do not limit the scope of the invention described in the claims.

### Example 1: Identification of Minimal Amino Acids in Cda2 Peptide

Use of full length recombinant proteins in T cell vaccine studies has the advantage that all epitopes are included in the antigen. However, identifying and defining the protective peptides contained within vaccine antigens could be beneficial. First, identifying immunodominant peptide regions of the protein allows elimination of regions of the protein that could drive non-essential, antagonistic, immune suppressive, or autoimmune responses. Second, using a synthesized peptide as a vaccine minimizes potentially confounding effects of extraneous vector (e.g., E. coli)-derived products, such as lipopolysaccharides, lipoproteins, and purification tags. Third, immunoprotective peptides could be combined into a chimeric recombinant protein which would simplify manufacturing and testing of a vaccine in clinical studies. In the present study, we performed an immunoinformatic analysis of Cda2 with the goal of defining CD4+ T cell epitopes for use in a Cryptococcus vaccine. Peptides within Cda2 were selected based on their predicted binding to the MHC Class II alleles of BALB/c and C57BL/6. Mutated peptides were then created to test the impact of MHC Class II binding.

### Methods

Reagents, Peptides, and C. neoformans. Except where noted, chemical reagents were obtained from Thermo Fisher Scientific. Peptides of >75% purity were synthesized by GenScript and provided as lyophilized material in measured amounts of peptide. Each peptide was analyzed by GenScript for purity using HPLC, Mass spectrometry and nitrogen analysis. Depending on their solubility, peptides were dissolved in water, 50% DMSO, or 100% DMSO. Stock solutions of each peptide were adjusted to 5 mg/ml based on their calculated extinction coefficient (E^{0.1%} at 280 nm; ProtParam tool at Expasy.org) and stored at −80° C. C. neoformans serotype A strain KN99α (41) was stored in glycerol stocks at −80° C. and grown for in vivo infection studies as described (15,16). Briefly, following an initial culture on YPD (Difco Yeast Extract, Bacto Peptone, Dextrose) with 2% agar, yeast cells were grown in liquid YPD at 30° C. with shaking for 18 h. Yeast cells were then harvested by centrifugation, washed with PBS, counted, and suspended in PBS at 2-4×10⁵ cells/ml.

Recombinant E. coli-expressed proteins. National Center for Biotechnology Information file for C. neoformans var. grubii H99 strain (taxid: 235443) served as the source for cDNA and protein sequences of Cda1 (CNAG_05799), Cda2 (CNAG_01230), Cda3 (CNAG_01239), and Fpd1 (CNAG_06291). cDNAs for these proteins and the mutated versions of Cda2 (Cda2-M1 and Cda2-M2) were synthesized and cloned in pET19b (GenScript) so that the vector-encoded His tag was integrated with the N-terminus of the cDNA. Recombinant protein was made in E. coli strain BL21(DE3) (New England BioLabs) using Overnite Express™ TB medium (Novagen) and purified on His Bind resin (EMD Millipore) in the presence of 6M urea, as described (16). Following elution with imidazole, proteins were dialyzed against 6M urea/20 mM Tris-HCl, pH7.9 and concentrated to 10 mg/ml using Amicon Ultra-15 centrifugal filters (10 kDA cutoff, Merck Millipore). The protein concentration was determined by the bicinchoninic acid (BCA) assay. To assess purity, the recombinant proteins were resolved on SDS-PAGE and stained with Coomassie InstantBlue (Expedeon, Ltd.).

GP-based vaccines. Recombinant E. coli-derived proteins were co-trapped with mouse serum albumin (MSA) complexed with yeast RNA (yRNA) in GPs as described (15, 16). Peptides which were water-soluble were loaded in an identical manner. Peptides in DMSO (5 mg/ml) were loaded by mixing 5 μl peptide per mg hydrated GPs, followed by lyophilization. DMSO (2.5 l/mg GPs) was added to then “push” the peptides into the core of the GPs, followed by lyophilization. A second “push” with 2.5 μl of water/mg GPs followed by lyophilization completed the loading of peptide. Subsequent steps were the same as was done for protein: MSA was loaded in 0.9% saline and the peptide/MSA inside the GPs were co-trapped with yRNA. Following the yRNA trapping step, peptide vaccines were sonicated to single particles, aliquoted, sonicated again and flash frozen. Protein vaccines were washed three times with saline before sonication. Vaccines were stored at −80° C. A vaccine dose consisted of 100 μl of 200 μg GPs (approximately 10⁸ particles) containing 10 μg of recombinant protein or 5 μg of synthesized peptide and 25 μg of MSA complexed with yRNA in 0.9% sterile saline. A control preparation, designated GP-MSA, contained MSA and yRNA without the antigen.

Mouse studies. C57BL/6, BALB/c, and Abb Knockout/Transgenic, HLA-DR4 (DR4) mice of both sexes were obtained from Charles River Laboratories, The Jackson Laboratory, and Taconic Biosciences. Mice were bred and housed in a specific pathogen-free environment in the animal facilities at the University of Massachusetts Chan Medical School (UMCMS). All animal procedures were carried out under a protocol approved by the UMCMS Institutional Use and Care of Animals Committee.

The vaccination and infection protocols were as described (15, 16). Briefly, vaccinations were administered subcutaneously three times at biweekly intervals. Mice received their first dose of vaccine when 6-10 weeks old. Two weeks following the last booster, the mice were anesthetized with isoflurane and challenged orotracheally with C. neoformans strain KN99α. The inoculum for DR4 and C57BL/6 mice was 1×10⁴ CFU while for BALB/c mice it was 2×10⁴ CFU. Mice were observed twice daily; humane endpoints prompting euthanasia included ataxia, listlessness, weight loss, and failure to groom. The experiment was terminated on day 70 post-infection at which time all survivors were euthanized.

Statistics. Kaplan-Meier survival curves were compared using the Mantel-Cox, log-rank test. The Bonferroni correction was applied in instances where multiple comparisons were made, with a P value of <0.05 considered significant after corrections were made. The software program GraphPad Prism Version 9.2.0 was used for all statistical analyses and to generate graphs.

Immunoinformatics. The immunoinformatics platform used has been described elsewhere (42, 43). Briefly, the mean and SD of natural log of ic50 MHC II allele binding for each sequential 15 amino acid peptide in the protein is predicted by artificial neural network ensembles using algorithms based on vectors derived from the principal components of the physical and chemical characteristics of each amino acid. Mean predicted binding is then standardized to a zero mean unit variance (normal) distribution within the protein to provide a relative competitive index of predicted binding for each peptide in the protein. This places binding predictions of all MHC alleles on the same scale. This metric is expressed in SD units relative to the mean for that protein. Comparison with other prediction systems indicates a predicted binding affinity of <−1 SD units below the mean is a probable epitope (44). The platform also evaluates cathepsin cleavage probability, and the frequency of any T cell exposed (non-pocket) motif relative to reference databases of the human proteome and bacteria of the gastrointestinal microbiome (45-47).

Alterations in the sequence of Cda2-Pep1 were designed to generate sequences with reduced H2-IAd binding affinity. This was done by generating 50,000 random iterations of Cda2-Pep1, progressively replacing 2-8 designated amino acids, and re-evaluating predicted binding of each constituent 15 amino acid peptide. A subset of peptides was then subjected to closer examination to select M1 and M2, each of which has diminished binding to H2-IAd at positions 203, 208 and/or 217. While the T cell exposed motifs in the region of interest are changed, there were no significant differences in the frequency of the exposed motifs relative to the reference databases, indicating that no obvious changes in T cell precursor frequency for the mutant peptides were created. FIG. 2 shows the differences in predicted binding of sequential 15 amino acid peptides within −M1 and −M2 compared to the original Cda2-Pep1 peptide.

### Results

Protection with the GP-Cda2 Protein Vaccine Varies as a Function of Mouse Strain.

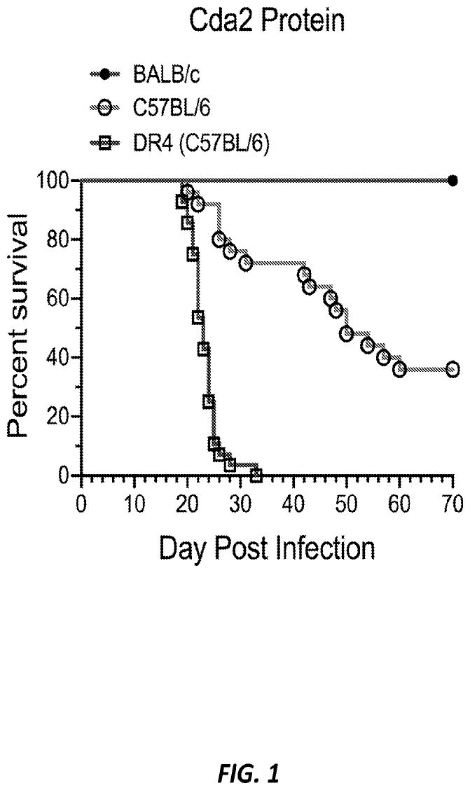

In FIG. 1, BALB/c (n=15), C57BL/6 (n=25), and DR4 (n=28) mice were vaccinated thrice with GP-Cda2 protein and then challenged with C. neoformans, as described in Methods section of Example 1 above. Mice were followed daily for survival until day 70 post infection. FIG. 1 includes mice previously published (16), as well as confirmatory new experiments. P<0.001 comparing any two groups. Not shown, survival of unvaccinated mice ranged from 20-32 days post infection for each of the mouse strains. Data demonstrate that a GP-based vaccine containing recombinant E. coli-derived Cda2 protect BALB/c mice more robustly than C57BL/6 mice (FIG. 1). Moreover, DR4 mice, which contain a humanized MHC II allele (DRB1*04:01) on a C57BL/6 genetic background are not significantly protected by the GP-Cda2 vaccine. This led us to hypothesize that the disparities in how well the GP-Cda2 vaccine protected the different mouse strains could be at least partially explained by differences in the MHC II molecules expressed. Our initial focus was on BALB/c mice, given the potent protection mediated by the GP-Cda2 vaccine in that mouse strain.

### Mutations in a Predicted High Binding Region of Cda2 Result in Substantial Loss of Vaccine-Mediated Protection in BALB c Mice.

FIGS. 2A-2D show the effect of mutations in a predicted high binding region of Cda2 on vaccine-mediated protection in BALB/c mice. We previously identified a region of Cda2 predicted to have 15 amino acid peptides with strong binding to H2-IAd, the MHC II allele expressed by BALB/c. This region also contains the amino acid sequence used to make a tetramer to identify Cda2-specific CD4 T cells following infection of C57BL/6 mice. We therefore created mutations in this region of Cda2 spanning amino acids 203-234 of Cda2 (FIG. 2A; mutated amino acids in Cda2-M1 and Cda2-M2 are indicated by arrows; mutations were designed as described in Methods.) so that on immunoinformatic analysis, the three predicted H2-IAd binding (designated by asterisks in FIG. 2B; predicted binding of each sequential 15mer peptides in CDA2 is shown by index (start) position of the peptide (X axis); the Y axis shows the predicted binding in standard deviation units relative to a mean of zero; a lower Standard Deviation (Z-Score) indicates greater predicted binding, as described in Methods) was greatly diminished or lost entirely. Two such mutated regions, designated M1 and M2 were selected. E. coli-derived proteins comprising these mutated sequences were then synthesized, and GP-based vaccines were manufactured. BALB/c mice were vaccinated, challenged via the pulmonary route with the KN99 strain of Cryptococcus, and followed for survival over a 70 d observation period. Vaccine-mediated protection was robust with recombinant “wild-type” Cda2 protein but was mostly lost when Cda2 proteins (Cda2-M1 and Cda2-M2) containing mutated sequences were used (FIG. 2C; control mice received GPs containing mouse serum albumin (MSA); the number of mice in each group: Cda2-Pep1, n=10; Cda2-M1, n=10; Cda2-M2, n=10; MSA, n=5).

### Protection Mediated by Peptide Vaccines Containing the Predicted High Binding Region of Cda2.

We synthesized 32-mer peptides spanning amino acids 203-234 from the predicted high binding region of Cda2, along with the corresponding regions of M1 and M2 mutants. These peptides were named Cda2-Pep1, Cda2-Pep1-M1, and Cda2-Pep1-M2, respectively. Remarkably, mice that received GP-based vaccines containing Cda2-Pep1 were protected from experimental cryptococcosis (FIG. 2D). In contrast, protection was diminished, albeit not eliminated, with the vaccines containing Cda2-Pep1-M1 and Cda2-Pep1-M2.

Protection Mediated by Vaccines Containing Peptides Homologous to Cda2-Pep1 that are Present in Other Cryptococcal Chitin Deacetylases.

FIGS. 3A-3B show protection mediated by GP-based vaccines containing peptides synthesized based on cryptococcal chitin deacetylases sequences with homology to the Cda2 predicted high binding region. Cda2 has homology to Cda1, Cda3 and Fpd1, including in the predicted MHCII IAd high binding region of Cda2 (FIG. 3A). We synthesized 32 amino acid peptides, termed Cda1-Pep1, Cda3-Pep1 and Fdp1-Pep1 based on sequences homologous to Cda2-Pep1. The peptides were loaded into GPs and used to vaccinate mice. Compared with unvaccinated mice, mice vaccinated with any of the GP-Pep1 vaccines were protected against an otherwise lethal pulmonary challenge with C. neoformans (FIG. 3B). Protection was greatest for vaccines containing Cda2-Pep1, followed by Cda1-Pep1, Cda3-Pep1, and Fpd1-Pep1.

### Protection of BALB/c and C57BL/6 Mice Mediated by GP-Based Vaccines Containing Other Peptide Sequences of Cda2.

We synthesized eight 31-35 amino acid peptides based on sequences in Cda2 (Table 2), loaded them into GPs, and tested the GP-peptide vaccines in BALB/c and C57BL/6 mouse models of cryptococcosis. FIGS. 4A-4D show protection of BALB/c and C57BL/6 mice mediated by GP-based vaccines containing Cda2 peptide sequences. The eight peptides were chosen to overlap with Cda2-Pep1 or based on regions in Cda2 predicted to contain good CD4⁺ T cell epitopes based on predicted binding to the H2-IAd allele in BALB/c (FIG. 4A; the peptides that were tested are shaded in gray; the y-axis shows the predicted binding in standard deviation units relative to a mean of zero; a lower Standard Deviation (Z-Score) indicates greater predicted binding, as described in Methods.) and/or the H2-IAb allele in C57BL/6 mice (FIG. 4C). Regarding the GP-peptide vaccines, compared with unvaccinated mice, in BALB/c mice, significant protection was seen in five of the eight vaccines (FIG. 4B; mice were followed daily for survival until day 70 post infection; each black dot represents one mouse, shown on the day post infection the mouse succumbed; the experiment was terminated 70 days post infection; survivors were assigned to day 70; the gray bars denote the geometric mean survival; UnVac=unvaccinated controls; peptide vaccines that afforded significant protection are shown in bold. P<0.0001 for Cda2-Pep1, Cda2-Pep2, Cda2-Pep3, and Cda2-Pep4. P=0.0003 for Cda2-Pep5). In contrast, of the eight peptide-based vaccines, only Cda2-Pep5 significantly protected C57BL/6 mice (FIG. 4D). Cda2-Pep5 includes what was predicted to be the strongest H2-IAb in the Cda2 recombinant protein (FIG. 4C).

CD4⁺ T cells are the most critical component of the adaptive protective immune response to naturally acquired cryptococcal infection. A challenge in developing cryptococcal vaccines has been the identification of antigens that induce protective CD4⁺ T cell responses, particularly given the diversity of MHC II in the human population (23). Herein, we performed an in depth study of an immunodominant protective protein antigen, Cda2, identifying regions of the protein contributing to vaccine-mediated protection in mice.

Our Cda2-derived peptide vaccines protected BALB/c more robustly than C57BL/6 mice. This is despite the protection afforded both mouse strains by the GP-based vaccine containing the E. coli-derived Cda2 protein. Cda2-Pep1, which was not protective as part of a GP-based vaccine, contains an epitope which is recognized by a sizable fraction of Th cells from infected C57BL/6 mice. This emphasizes that immunogenicity does not necessarily correlate with protection. BALB/c mice are relatively resistant to cryptococcal infection compared with C57BL/6 mice. This effect has been attributed in part to a protective Th1 response developing in BALB/c, whereas C57BL/6 mice develop a Th2-biased response. While the GP vaccine platform skews towards Th1- and Th17-type responses, a response that is broader than just to a single peptide may be required to protect C57BL/6 mice. In addition, the possible contributions of antibody and CD8⁺ T cell immunity must be considered. An alternate but not mutually exclusive possible explanation is the site in Cda2, which comprises the BALB/c MHCII IAd binding site has a functional role that is targeted by the immune response. Of note, Cda2-Pep1 contains two conserved histidines required for metal-binding in the catalytic domain of fungal chitin deacetylases and bacterial peptidoglycan deacetylases.

Cda1, Cda2, Cda3, and Fpd1 define a family of homologous chitin deacetylases responsible for deacetylating chitin to chitosan in the cryptococcal cell wall. Homology is particularly high in the region contained within Cda2-Pep1, our most protective peptide. GP-based peptide vaccines containing regions in Cda1, Cda2, and Fdp1 homologous to Cda2-Pep1 protected BALB/c mice against cryptococcal challenge.

MHC binding predictions focus on the flanking regions of the T cell epitopes, also called the pocket positions. The binding affinity indicates the quantitative relationship of a potential epitope with the cognate T cells, based on the on-off rate of the T cell receptor and hence the frequency of interactions between the T cell and epitope. Conversely, the amino acid motifs actually engaging a T cell receptor (the non-pocket residues or T cell-exposed motif) are a qualitative interaction. The T cell-exposed motifs are comprised of the central amino acids of any of the 15 amino acid peptides, typically a discontinuous pentamer comprising positions 2, 3, 5, 7, 8 of the central 9 amino acid core. While there is considerable homology between the sequences from the four proteins we have examined, there is also sufficient sequence diversity that the T cell exposed motifs are different between the 4 proteins. Only Cda1-Pep1 and Cda3-Pep1 share exact identity in just one T cell-exposed motif Any cross-reactivity among the proteins would depend on “near neighbor” binding of T cells to similar, but non-identical, motifs.

Our data serve as a proof of principle that peptide vaccines engineered to stimulate CD4⁺ T cell responses can protect mice against a highly virulent C. neoformans strain. The vaccines were adjuvanted and delivered using the GP platform, which biases towards strong Th1 and Th17 responses; future studies will be needed to determine whether other adjuvants can be substituted. Given the diversity of MHC II alleles in the human population, a peptide-based vaccine designed for use in humans would likely require multiple peptides. An additional challenge to translate our findings to humans is the impairments in CD4⁺ T cell function present in most individuals at risk for cryptococcosis. Individuals would likely need to be vaccinated when their T cell function was relatively intact, such as early in HIV infection or prior to solid organ transplantation. Moreover, combining a T cell vaccine with one that elicits protective antibodies merits testing.

### REFERENCES FOR EXAMPLE 1

1. Heitman J, Kozel T R, Kwon-Chung K J, Perfect J R, Casadevall A (ed). 2011. Cryptococcus: From Human Pathogen to Model Yeast. ASM Press, Washington DC, 2010.
2. Brown G D, Denning D W, Gow N A, Levitz S M, Netea M G, White T C. 2012. Hidden killers: human fungal infections. Science translational medicine 4:165rv13.
3. Rajasingham R, Smith R M, Park B J, Jarvis J N, Govender N P, Chiller T M, Denning D W, Loyse A, Boulware D R. 2017. Global burden of disease of HIV-associated cryptococcal meningitis: an updated analysis. The Lancet infectious diseases 17:873-881.
4. Singh N, Dromer F, Perfect J R, Lortholary O. 2008. Cryptococcosis in solid organ transplant recipients: current state of the science. Clin Infect Dis 47:1321-7.
5. Wozniak K L, Levitz S M. 2011. T Cell and Dendritic Cell Immune Responses to Cryptococcus, p 387-396. In Heitman J, Kozel T R, Kwon-Chung J K, Perfect J R, Casadevall A (ed), Cryptococcus: From Human Pathogen to Model Yeast. ASM Press, Washington, D.C.
6. Ueno K, Yanagihara N, Shimizu K, Miyazaki Y. 2020. Vaccines and Protective Immune Memory against Cryptococcosis. Biol Pharm Bull 43:230-239.
7. Abraham A, Ostroff G, Levitz S M, Oyston P C F. 2019. A novel vaccine platform using glucan particles for induction of protective responses against Francisella tularensis and other pathogens. Clin Exp Immunol 198:143-152.
8. Wang Y, Wang K, Masso-Silva J A, Rivera A, Xue C. 2019. A Heat-Killed Cryptococcus Mutant Strain Induces Host Protection against Multiple Invasive Mycoses in a Murine Vaccine Model. mBio 10:02145.
9. Colombo A C, Rella A, Normile T, Joffe L S, Tavares P M, de S. Aranjo G R, Frases S, Omer E P, Farnoud A M, Fries B C, Sheridan B, Nimrichter L, Rodrigues M L, Del Poeta M. 2019. Cryptococcus neoformans Glucuronoxylomannan and Sterylglucoside Are Required for Host Protection in an Animal Vaccination Model. mBio 10:e02909-18.
10. Zhai B, Wozniak K L, Masso-Silva J, Upadhyay S, Hole C, Rivera A, Wormley F L, Lin X. 2015. Development of Protective Inflammation and Cell-Mediated Immunity against Cryptococcus neoformans after Exposure to Hyphal Mutants. mBio 6:e01433-15.
11. Upadhya R, Lam W C, Maybruck B, Specht C A, Levitz S M, Lodge J K. 2016.

Induction of Protective Immunity to Cryptococcal Infection in Mice by a Heat-Killed, Chitosan-Deficient Strain of Cryptococcus neoformans. mBio 7: e00547-16.
12. Wormley F L, Jr., Perfect J R, Steele C, Cox G M. 2007. Protection against cryptococcosis by using a murine gamma interferon-producing Cryptococcus neoformans strain. Infection and immunity 75:1453-62.
13. Van Dyke M C C, Chaturvedi A K, Hardison S E, Leopold Wager C M, Castro-Lopez N, Hole C R, Wozniak K L, Wormley F L. 2017. Induction of Broad-Spectrum Protective Immunity against Disparate Cryptococcus Serotypes. Frontiers in Immunology 8:1-16.
14. Levitz S M, Golenbock D T. 2012. Beyond empiricism: informing vaccine development through innate immunity research. Cell 148:1284-92.
15. Hester M M, Lee C K, Abraham A, Khoshkenar P, Ostroff G R, Levitz S M, Specht C A. 2020. Protection of mice against experimental cryptococcosis using glucan particle-based vaccines containing novel recombinant antigens. Vaccine 38:620-626.
16. Specht C A, Lee C K, Huang H, Hester M M, Liu J, Luckie B A, Torres Santana M A, Mirza Z, Khoshkenar P, Abraham A, Shen Z T, Lodge J K, Akalin A, Homan J, Ostroff G R, Levitz S M. 2017. Vaccination with Recombinant Cryptococcus Proteins in Glucan Particles Protects Mice against Cryptococcosis in a Manner Dependent upon Mouse Strain and Cryptococcal Species. MBio 8:e01872-17.
17. Levitz S M, Nong S, Mansour M K, Huang C, Specht C A. 2001. Molecular characterization of a mannoprotein with homology to chitin deacetylases that stimulates T cell responses to Cryptococcus neoformans. Proc Natl Acad Sci USA 98:10422-7.
18. Wiesner D L, Specht C A, Lee C K, Smith K D, Mukaremera L, Lee S T, Lee C G, Elias J A, Nielsen J N, Boulware D R, Bohjanen P R, Jenkins M K, Levitz S M, Nielsen K. 2015. Chitin recognition via chitotriosidase promotes pathologic type-2 helper T cell responses to cryptococcal infection. PLoS pathogens 11:e1004701.
19. Baker L G, Specht C A, Lodge J K. 2011. Cell wall chitosan is necessary for virulence in the opportunistic pathogen Cryptococcus neoformans. Eukaryotic cell 10:1264-8.
20. Hembach L, Bonin M, Gorzelanny C, Moerschbacher B M. 2020. Unique subsite specificity and potential natural function of a chitosan deacetylase from the human pathogen Cryptococcus neoformans. Proceedings of the National Academy of Sciences 117:3551-3559.
21. Hurtgen B J, Hung C Y, Ostroff G R, Levitz S M, Cole G T. 2012. Construction and evaluation of a novel recombinant T cell epitope-based vaccine against Coccidioidomycosis. Infect Immun 80:3960-74.
22. Bremel R D, Homan E J. 2015. Extensive T-Cell Epitope Repertoire Sharing among Human Proteome, Gastrointestinal Microbiome, and Pathogenic Bacteria: Implications for the Definition of Self. Front Immunol 6:538.
23. Oliveira L V N, Wang R, Specht C A, Levitz S M. 2021. Vaccines for human fungal diseases: close but still a long way to go. NPJ Vaccines 6:33.
24. Huffnagle G B, Boyd M B, Street N E, Lipscomb M F. 1998. IL-5 Is Required for Eosinophil Recruitment, Crystal Deposition, and Mononuclear Cell Recruitment During a Pulmonary Cryptococcus neoformans Infection in Genetically Susceptible Mice (C57BL/6). The Journal of Immunology 160:2393-2400.
25. Chen G-h, McNamara D A, Hernandez Y, Huffnagle G B, Toews G B, Olszewski M A. 2008. Inheritance of Immune Polarization Patterns Is Linked to Resistance versus Susceptibility to Cryptococcus neoformans in a Mouse Model. Infect Immun 76:2379-2391.
26. Huang H, Ostroff G R, Lee C K, Agarwal S, Ram S, Rice P A, Specht C A, Levitz S M. 2012. Relative contributions of dectin-1 and complement to immune responses to particulate beta-glucans. J Immunol 189:312-7.
27. Huang H, Ostroff G R, Lee C K, Specht C A, Levitz S M. 2013. Characterization and optimization of the glucan particle-based vaccine platform. Clin Vaccine Immunol 20:1585-91.
28. Upadhya R, Baker L G, Lam W C, Specht C A, Donlin M J, Lodge J K. 2018. Cryptococcus neoformans Cda1 and Its Chitin Deacetylase Activity Are Required for Fungal Pathogenesis. mBio 9.
29. Blair D E, Hekmat O, Schuttelkopf A W, Shrestha B, Tokuyasu K, Withers S G, van Aalten D M. 2006. Structure and mechanism of chitin deacetylase from the fungal pathogen Colletotrichum lindemuthianum. Biochemistry 45:9416-26.
30. Petrova G, Ferrante A, Gorski J. 2012. Cross-reactivity of T cells and its role in the immune system. Critical reviews in immunology 32:349-372.
31. Gutierrez A H, Loving C, Moise L, Terry F E, Brockmeier S L, Hughes H R, Martin W D, De Groot A S. 2016. In Vivo Validation of Predicted and Conserved T Cell Epitopes in a Swine Influenza Model. PLoS One 11:e0159237.
32. Li T, Steede N K, Nguyen H N, Freytag L C, McLachlan J B, Mettu R R, Robinson J E, Landry S J. 2014. Comprehensive analysis of contributions from protein conformational stability and major histocompatibility complex class II-peptide binding affinity to CD4+ epitope immunogenicity in HIV-1 envelope glycoprotein. J Virol 88:9605-15.
33. Oli A N, Obialor W O, Ifeanyichukwu M O, Odimegwu D C, Okoyeh J N, Emechebe G O, Adejumo S A, Ibeanu G C. 2020. Immunoinformatics and Vaccine Development: An Overview. Immunotargets Ther 9:13-30.
34. Rappuoli R, Bottomley M J, D'Oro U, Finco O, De Gregorio E. 2016. Reverse vaccinology 2.0: Human immunology instructs vaccine antigen design. J Exp Med 213:469-81.
35. Levitz S M, Specht C A. 2006. The molecular basis for the immunogenicity of Cryptococcus neoformans mannoproteins. FEMS Yeast Res 6:513-24.
36. Specht C A, Nong S, Dan J M, Lee C K, Levitz S M. 2007. Contribution of glycosylation to T cell responses stimulated by recombinant Cryptococcus neoformans mannoprotein. J Infect Dis 196:796-800.
37. Zhou J Y, Oswald D M, Oliva K D, Kreisman L S C, Cobb B A. 2018. The Glycoscience of Immunity. Trends Immunol 39:523-535.
38. Deepe G S, Jr., Buesing W R, Ostroff G R, Abraham A, Specht C A, Huang H, Levitz S M. 2018. Vaccination with an alkaline extract of Histoplasma capsulatum packaged in glucan particles confers protective immunity in mice. Vaccine 36:3359-3367.
39. Specht C A, Lee C K, Huang H, Tipper D J, Shen Z T, Lodge J K, Leszyk J, Ostroff G R, Levitz S M. 2015. Protection against Experimental Cryptococcosis following Vaccination with Glucan Particles Containing Cryptococcus Alkaline Extracts. mBio 6:e01905-15.
40. Casadevall A, Feldmesser M, Pirofski L-a. 2002. Induced humoral immunity and vaccination against major human fungal pathogens. Current opinion in microbiology 5:386-391.
41. Nielsen K, Cox G M, Wang P, Toffaletti D L, Perfect J R, Heitman J. 2003.

Sexual cycle of Cryptococcus neoformans var. grubii and virulence of congenic a and alpha isolates. Infection and immunity 71:4831-41.
42. Bremel R D, Homan E J. 2010. An integrated approach to epitope analysis II: A system for proteomic-scale prediction of immunological characteristics. ImmunomeRes 6:8.
43. Bremel R D, Homan E J. 2013. Recognition of higher order patterns in proteins: immunologic kernels. PLoS One 8:e70115.
44. Homan E J, Bremel R D. 2011. Patterns of Predicted T-Cell Epitopes Associated with Antigenic Drift in Influenza H3N2 Hemagglutinin. PLoSOne 6:e26711.
45. Bremel R D, Homan E J. 2014. Frequency Patterns of T-Cell Exposed Amino Acid Motifs in Immunoglobulin Heavy Chain Peptides Presented by MHCs. Front Immunol 5:541.
46. Bremel R D, Homan J. 2015. Extensive T-cell epitope repertoire sharing among human proteome, gastrointestinal microbiome, and pathogenic bacteria: Implications for the definition of self. Frontiers in Immunology 6.
47. Hoglund R A, Torsetnes S B, Lossius A, Bogen B, Homan E J, Bremel R, Holmoy T. 2019. Human Cysteine Cathepsins Degrade Immunoglobulin G In Vitro in a Predictable Manner. Int J Mol Sci 20.

### Example 2: CAF01 as an Adjuvant

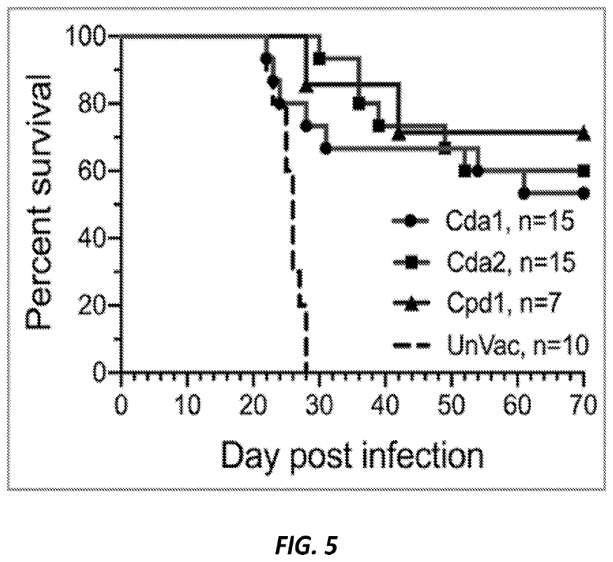

Adjuvants increase the quantity and direct the quality of a vaccine immune response. Promising protection data has been obtained using GPs as a combined adjuvant and delivery system. However, large scale manufacture of GP-based cryptococcal vaccines could be too expensive for use in the resource-poor settings where they are most needed. Also, in the absence of large clinical studies, the reactogenicity of GP-based vaccines remains unknown. Thus, to maximize our chances of bringing a successful vaccine forward, we tested our Ags with adjuvants other than GPs. In preliminary studies (data not shown), the following adjuvants afforded no protection (100% mortality at d30 post fungal challenge) when formulated with Cda1 and Cda2: alum (Imject), Adjuplex, CRX-527 (TLR4 ligand), UM-1007 (TLR7/8 ligand), and UM1052 (Mincle ligand). However, immunization with Cda1, Cda2 and Cpd1 in CAF01 afforded significant protection with BALB/c mice (FIG. 5; mice received a prime and two biweekly boosts with 10 μg of the indicated protein formulated in CAF01; two weeks after the last boost, mice were challenged with 2×10⁴ Cn KN99α and followed 70 days for survival; number of mice/group=15, 15, 7 and 10 for Cda1, Cda2, Cpd1 and unvaccinated (UnVac). p<0.001 comparing any of the vaccinated groups with UnVac (unvaccinated)). Not shown on FIG. 5, for C57BL/6 mice vaccinated Cda1 and Cda2 in CAF01, there was 60% and 40% survival at 70 d, respectively, versus 0% survival in unvaccinated mice (p<0.001, n=10 mice/group).

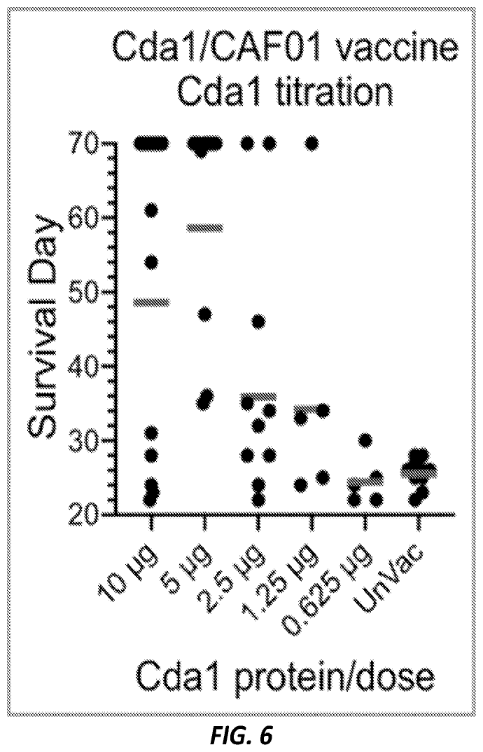

Regarding the optimal amount of antigen, FIG. 6 demonstrates that when adjuvanted in CAF01, 5-10 μg of Cda1 is optimal. Mice received a prime and two biweekly boosts with the indicated amount of recombinant Cda1 protein formulated in CAF01. Two weeks after the last boost, mice received a pulmonary challenge (i.e., were challenged with 2×10⁴ Cn KN99α) and followed 70 days for survival. Each dot represents one mouse. The horizontal bars represent geometric mean survival for each group. Survival day refers to the day the mouse succumbed to infection. Mice surviving 70 days post infection were assigned 70 as their survival day. UnVac; unvaccinated. The optimal antigen concentration appears to be 5-10 μg per vaccine dose.

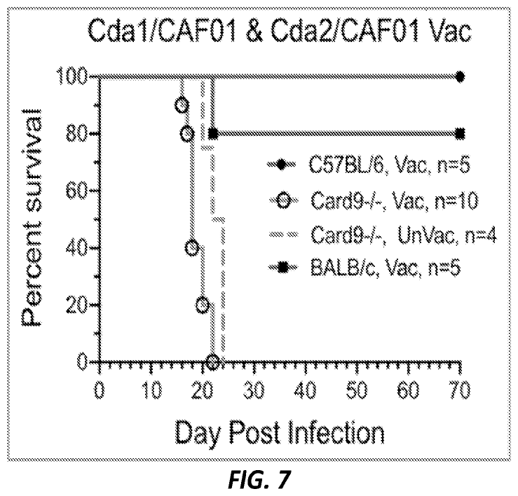

In FIG. 7, mice were vaccinated with a combination of Cda1-CAF01 and Cda2-CAF01. The mice received a prime and two booster shots of each vaccine, two weeks apart. Mice were then challenged via the pulmonary route with C. neoformans and followed for survival. We first compared vaccine-mediated protection in C57BL/6 mice with BALB/c mice. We found that protection was comparable in the two mouse strains. Also shown in FIG. 7, we looked at vaccine-mediated protection in mice genetically deficient in the adapter protein, CARD9. The rationale for examining Card9−/− mice is Mincle uses the adapter protein CARD9 to signal. As noted above, CAF01 is a Mincle agonist. We found that mice lacking CARD9 were no longer protected by the CAF01.

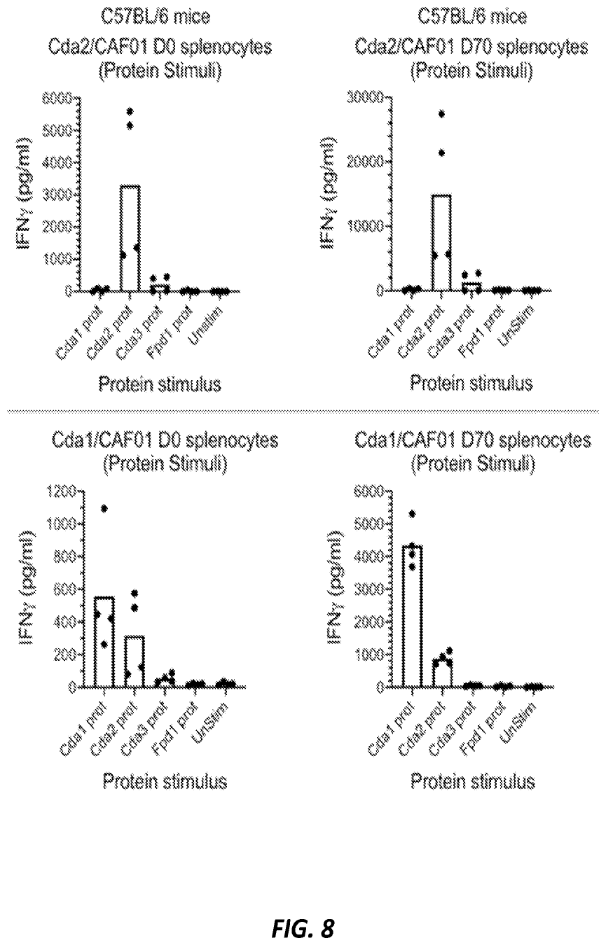

To further examine the immunological mechanisms of protection mediated by CAF01-adjuvanted vaccines, we performed ex vivo stimulation assays of Cda2-CAF01 and Cda1-CAF01 vaccinated mice. Mice received a prime and two biweekly boosts with 10 μg of recombinant Cda2 (top panels) or Cda1 (bottom panels) protein formulated in CAF01. Two weeks after the last boost, some mice (designated DO for day 0) were euthanized and their spleens harvested. Other mice were challenged via the pulmonary route with C. neoformans; for these mice, spleens were harvested from 70 day survivors (designated D70). Splenocytes were stimulated with the indicated recombinant cryptococcal and then interferon-gamma (IFNγ) was measured in the supernatants by ELISA. Each dot represents values from an individual mouse. The top set of experiments in FIG. 8 is with the Cda2-CAF01 vaccine whereas the bottom set in FIG. 8 is with the Cda1-CAF01 vaccine. Note that with both vaccines, there was a very strong interferon response to the vaccine protein. Cda1, Cda2, Cda3 and Fpd1 share some homology; this likely accounts for the response to Cda2 in the Cda1-vaccinated mice.

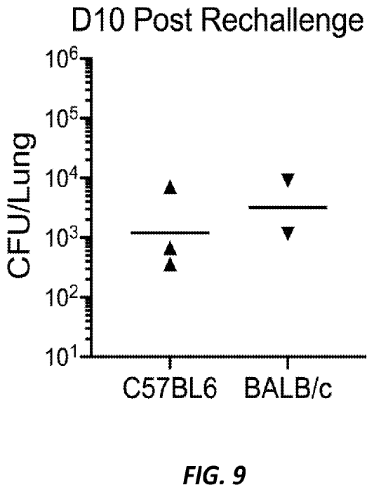

In another experiment, we looked at the durability of vaccine-mediated protection. BALB/c and C57BL/6 mice were vaccinated with Cda1-CAF01 and Cda2-CAF01. The vaccines were administered into separate sites subcutaneously and given biweekly times three doses, with 10 μg protein per vaccine. Two weeks after the last booster vaccine, mice were challenged with C. neoformans. Surviving mice at day 168 were then rechallenged with 2×10⁴ CFU C. neoformans. Mice were euthanized 10 days post rechallenge and lung CFU were measured. Each triangle represents one mouse. There were 5 mice from each strain; all the mice survived 168 days post-infection except for one BALB/c mouse. Thus, the CAF01-containing vaccines can mediate long-lasting protection. We then rechallenged these vaccinated mice with C. neoformans; 10 days post-rechallenge the mice were euthanized. Microbiology and immunology experiments were performed on harvested lungs. Lung colony forming units were lower than in the inoculum of 2×10⁴ CFU (FIG. 9), demonstrating partial clearance. In FIG. 9, BALB/c and C57BL/6 mice were vaccinated with Cda1-CAF01 and Cda2-CAF01. The vaccines were administered into separate sites subcutaneously and given biweekly times three doses, with 10 μg protein per vaccine. Two weeks after the last vaccine, mice were challenged with C. neoformans.

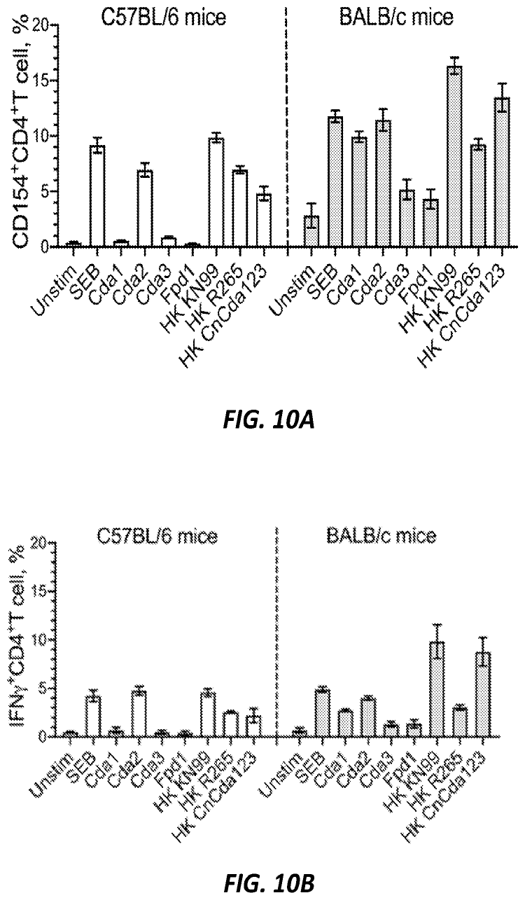

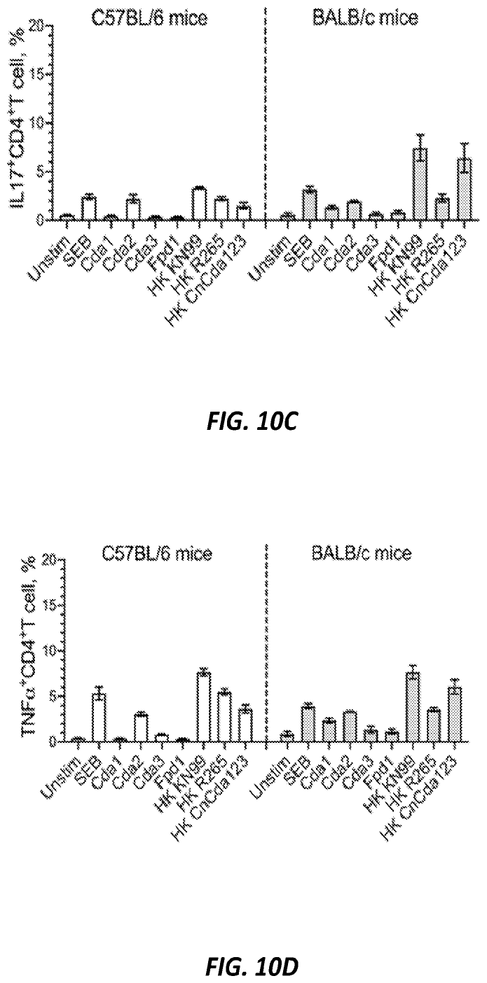

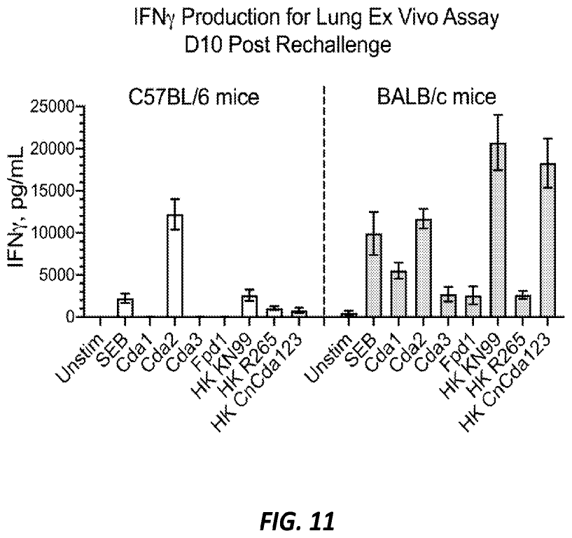

For the immunology experiments, single cell lung suspensions were prepared and cultured ex vivo for 18 hours in the presence of the stimuli indicated in FIGS. 10A-10D. BALB/c and C57BL/6 mice were vaccinated with Cda1-CAF01 and Cda2-CAF01. The vaccines were administered into separate sites subcutaneously and given biweekly times three doses, with 10 μg protein per vaccine. Two weeks after the last vaccine, mice were challenged with C. neoformans. Surviving mice at day 168 were then rechallenged with 2×10⁴ CFU C. neoformans. Mice were euthanized 10 days post rechallenge. Single cell lung suspensions were made, and the cells were stimulated for 18 hours with the indicated stimuli. Brefeldin A was added for the last 7 hours of the incubation to inhibit cytokine secretion. Lung CD4 T cells were permeabilized and analyzed by flow cytometry with intracellular staining for the activation marker CD154 and the intracellular cytokines IFNγ, IL-17, and TNFα. Unstim, unstimulated cells. SEB, Staphylococcal enterotoxin B (a superantigen serving as a positive control). HK KN99, HK R265 and HK CnCda123 are heat-killed strains of Cryptococcus. Flow cytometry with intracellular cytokine staining was then performed on gated CD4+ T cells. Activated T cells were determined by expression of CD154. Intracellular cytokines measured were interferon gamma (IFNγ, a marker of Th1 cells), IL-17a (a marker of Th17 cells) and tumor necrosis factor alpha (TNFα). Importantly, IFNγ, IL-17 and TNFα have each been associated with protection from cryptococcosis. We found that the vaccinated mice had robust antigen-specific stimulation of CD4+ T cells which expressed CD154+, IFNγ, IL-17, and TNFα. Note that Cda1, Cda2, Cda3 and Fdp1 are homologous members of the chitin deacetylase family, which might account for some of the cross-reactivity observed. We also measured IFNγ in the supernatants of the ex vivo stimulated cells by ELISA (FIG. 11; note that the methods are the same as in FIGS. 10A-10D except supernatants were analyzed for IFNγ following the 18 hour ex vivo stimulation.). The ELISA data mainly paralleled what was found by flow cytometry for IFNγ-expressing CD4+ T cells in that antigen-specific stimulation of IFNγ was observed.

### REFERENCES FOR EXAMPLE 2

1. Hester M M, Lee C K, Abraham A, Khoshkenar P, Ostroff G R, Levitz S M, et al. Protection of mice against experimental cryptococcosis using glucan particle-based vaccines containing novel recombinant antigens. Vaccine. 2020; 38(3):620-6.
2. Specht C A, Lee C K, Huang H, Hester M M, Liu J, Luckie B A, et al. Vaccination with Recombinant Cryptococcus Proteins in Glucan Particles Protects Mice against Cryptococcosis in a Manner Dependent upon Mouse Strain and Cryptococcal Species. MBio. 2017; 8(6):e01872-17.
3. Huang H, Ostroff G R, Lee C K, Agarwal S, Ram S, Rice P A, et al. Relative contributions of dectin-1 and complement to immune responses to particulate beta-glucans. J Immunol. 2012; 189(1):312-7.
4. Huang H, Ostroff G R, Lee C K, Specht C A, Levitz S M. Robust stimulation of humoral and cellular immune responses following vaccination with antigen-loaded beta-glucan particles. mBio. 2010; 1(3).
5. Huang H, Ostroff G R, Lee C K, Specht C A, Levitz S M. Characterization and optimization of the glucan particle-based vaccine platform. Clin Vaccine Immunol. 2013; 20(10):1585-91.
6. Levitz S M, Huang H, Ostroff G R, Specht C A. Exploiting fungal cell wall components in vaccines. Semin Immunopathol. 2015; 37(2):199-207.
7. Specht C A, Lee C K, Huang H, Tipper D J, Shen Z T, Lodge J K, et al. Protection against Experimental Cryptococcosis following Vaccination with Glucan Particles Containing Cryptococcus Alkaline Extracts. mBio. 2015; 6(6):e01905-15.
8. Luberto C, Martinez-Marino B, Taraskiewicz D, Bolanos B, Chitano P, Toffaletti D L, et al. Identification of Appl as a regulator of phagocytosis and virulence of Cryptococcus neoformans. J Clin Invest. 2003; 112(7):1080-94.
9. Park Y D, Shin S, Panepinto J, Ramos J, Qiu J, Frases S, et al. A role for LHC1 in higher order structure and complement binding of the Cryptococcus neoformans capsule. PLoS Pathog. 2014; 10(5):e1004037.
10. Levitz S M, Golenbock D T. Beyond empiricism: informing vaccine development through innate immunity research. Cell. 2012; 148(6):1284-92.
11. Coffman R L, Sher A, Seder R A. Vaccine Adjuvants: Putting Innate Immunity to Work. Immunity. 2010; 33(4):492-503.
12. Wegmann F, Moghaddam A E, Schiffner T, Gartlan K H, Powell T J, Russell R A, et al. The Carbomer-Lecithin Adjuvant Adjuplex Has Potent Immunoactivating Properties and Elicits Protective Adaptive Immunity against Influenza Virus Challenge in Mice. Clinical and vaccine immunology: CVI. 2015; 22(9):1004-12.
13. Gasper D J, Neldner B, Plisch E H, Rustom H, Carrow E, Imai H, et al. Effective Respiratory CD8 T-Cell Immunity to Influenza Virus Induced by Intranasal Carbomer-Lecithin-Adjuvanted Non-replicating Vaccines. PLoS pathogens. 2016; 12(12):e1006064-e.
14. Cluff C W, Baldridge J R, Stover A G, Evans J T, Johnson D A, Lacy M J, et al. Synthetic Toll-Like Receptor 4 Agonists Stimulate Innate Resistance to Infectious Challenge. Infection and Immunity. 2005; 73(5):3044-52.
15. Tan Z Y, Khah A K L, Sim S H, Novem V, Liu Y, Tan G-Y G. Synthetic TLR4 agonist as a potential immunotherapy for melioidosis. Open Journal of Immunology. 2013; Vol. 03No. 01:9.
16. Smith A J, Miller S M, Buhl C, Child R, Whitacre M, Schoener R, et al. Species-Specific Structural Requirements of Alpha-Branched Trehalose Diester Mincle Agonists. Frontiers in Immunology. 2019; 10(338).
17. Christensen D, Mortensen R, Rosenkrands I, Dietrich J, Andersen P. Vaccine-induced Th17 cells are established as resident memory cells in the lung and promote local IgA responses. Mucosal Immunol. 2017; 10(1):260-70.
18. Lindenstrom T, Agger E M, Korsholm K S, Darrah P A, Aagaard C, Seder R A, et al. Tuberculosis subunit vaccination provides long-term protective immunity characterized by multifunctional CD4 memory T cells. J Immunol. 2009; 182(12):8047-55.
19. Lindenstrom T, Woodworth J, Dietrich J, Aagaard C, Andersen P, Agger E M. Vaccine-Induced Th17 Cells Are Maintained Long-Term Postvaccination as a Distinct and Phenotypically Stable Memory Subset. Infection and Immunity. 2012; 80(10):3533-44.
20. Schick J, Schafer J, Alexander C, Dichtl S, Murray P J, Christensen D, et al. Cutting Edge: TNF Is Essential for Mycobacteria-Induced MINCLE Expression, Macrophage Activation, and Th17 Adjuvanticity. The Journal of Immunology. 2020; 205(2):323-8.
21. Pedersen G K, Andersen P, Christensen D. Immunocorrelates of CAF family adjuvants. Semin Immunol. 2018; 39:4-13.
22. Desel C, Werninghaus K, Ritter M, Jozefowski K, Wenzel J, Russkamp N, et al. The Mincle-activating adjuvant TDB induces MyD88-dependent Th1 and Th17 responses through IL-1R signaling. PLoS One. 2013; 8(1):e53531.
23. Abraham S, Juel H B, Bang P, Cheeseman H M, Dohn R B, Cole T, et al. Safety and immunogenicity of the chlamydia vaccine candidate CTH522 adjuvanted with CAF01 liposomes or aluminium hydroxide: a first-in-human, randomised, double-blind, placebo-controlled, phase 1 trial. Lancet Infect Dis. 2019; 19(10):1091-100.
24. van Dissel J T, Joosten S A, Hoff S T, Soonawala D, Prins C, Hokey D A, et al. A novel liposomal adjuvant system, CAF01, promotes long-lived Mycobacterium tuberculosis-specific T-cell responses in human. Vaccine. 2014; 32(52):7098-107.
25. Wozniak K L, Hardison S E, Kolls J K, Wormley F L. Role of IL-17A on resolution of pulmonary C. neoformans infection. PLoS One. 2011; 6(2):e17204.
26. Herring A C, Lee J, McDonald R A, Toews G B, Huffnagle G B. Induction of Interleukin-12 and Gamma Interferon Requires Tumor Necrosis Factor Alpha for Protective T1-Cell-Mediated Immunity to Pulmonary Cryptococcus neoformans Infection. Infect Immun. 2002; 70(6):2959-64.
27. Kawakami K, Qifeng X, Tohyama M, Qureshi M H, Saito A. Contribution of tumour necrosis factor-alpha (TNF-alpha) in host defence mechanism against Cryptococcus neoformans. Clinical and Experimental Immunology. 1996; 106(3):468-74.
28. Wiesner D L, Specht C A, Lee C K, Smith K D, Mukaremera L, Lee S T, et al. Chitin recognition via chitotriosidase promotes pathologic type-2 helper T cell responses to cryptococcal infection. PLoS Pathogens. 2015; 11(3):e1004701.

### Example 3: Vaccine-Mediated Protection Following T Cell Immunocompromise

As most cases of cryptococcosis occur in the setting of T cell dysfunction, a cryptococcal vaccine should be designed to protect these high risk individuals. There are opportunities to vaccinate patients when T cell dysfunction is not severe, such as an HIV-infected patient with CD4⁺ T cell counts above 100/μL blood or a patient on a transplant waiting list. It would be hoped that as immunocompromise worsens, some protection would remain. Moreover, many (if not most) cases of cryptococcosis are due to reactivation of latent infection (90); vaccination could reduce the fungal burden in those persons.

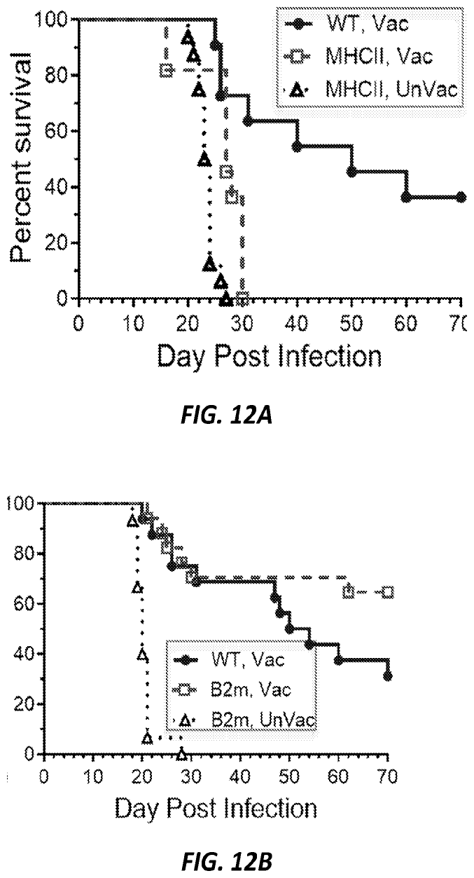

To examine the role of T cells in protection, wildtype (WT), MHC class II^{-/-} (MHCII^{-/-}; CD4-deficient) and β-2-Microglobulin^{-/-} (β2m^{-/-}; CD8-deficient) mice were vaccinated with GP-Cda2, challenged with Cn strain KN99α and followed for survival. Mice lacking CD4⁺ T cells had 100% mortality by d30, analogous to that seen for unvaccinated mice (FIG. 12A; wild-type (WT) C57BL/6 mice and CD4 T cell-deficient (MHCII) mice on the C57BL/6 background received a prime and two biweekly subcutaneous boosts with GP-Cda2; one week after the last boost, mice were challenged with 1×10⁴ Cn KN99α and followed for 70 days for survival; Vac=vaccinated with GP-Cda2; UnVac—unvaccinated). In contrast, protection was retained in CD8-deficient mice; if anything, there was a non-significant trend towards enhanced survival (FIG. 12B; same as FIG. 12B except CD8 T cell-deficient (B2m) mice were used; all experiments were repeated at least once with 11-17 mice/group. P<0.001 comparing WT,Vac and MHCII,Vac (Mantel-Cox log rank test)). Of note when interpreting these results, β2m-deficient mice have perturbations aside from CD8 deficiency, including high iron levels. Similar results were seen for the CD4^{-/-} and CD8^{-/-} mice vaccinated with GP-Cda1 and GP-Blp4; protection was completely abolished in the presence of CD4 deficiency (100% mortality by d30) but not significantly affected by CD8 deficiency (data not shown).

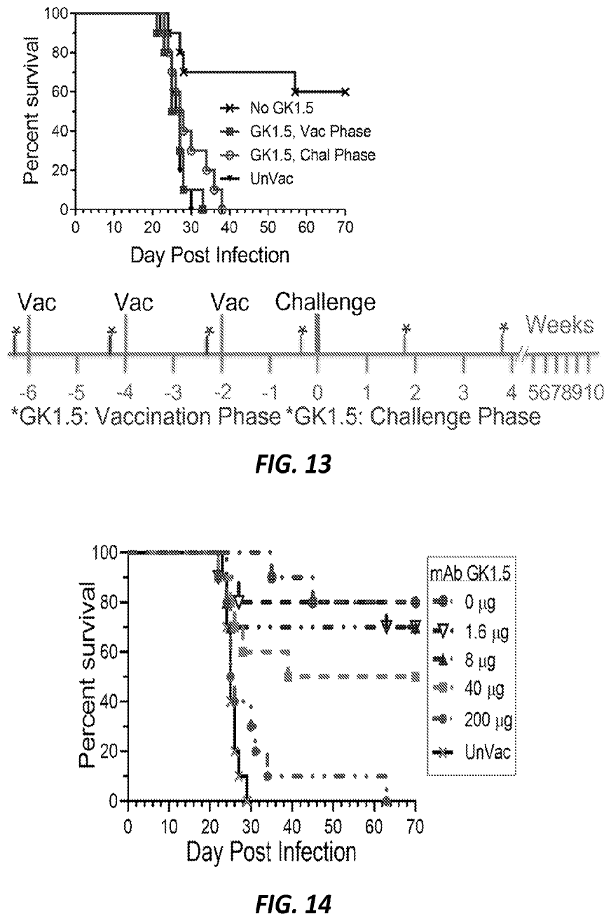

The importance of CD4⁺ T cells in GP vaccine-mediated protection was also demonstrated by antibody depletion using the anti-CD4 monoclonal antibody (mAb) GK1.5. In preliminary studies (not shown), each injection of 200 μg of GK1.5 led to nearly complete depletion of blood CD4 T cells for at least 2 weeks. Two strategies of CD4 depletion were used. In the first, mice were depleted of CD4⁺ cells in the vaccination phase by giving a dose of GK1.5 two days prior to each vaccination. For the second strategy, GK1.5 was given two days prior to fungal challenge and then two additional biweekly injections were given (FIG. 13; BALB/c mice were vaccinated (vac) subcu with GP-Cda2 as indicated and then given a challenge dose of 2×10⁴ Cn KN99α and followed for 70 days for survival; the CD4-depleting mAb GK1.5 was injected at three biweekly intervals either during the vaccination phase (Vac Phase) or the challenge phase (Chal Phase); controls including vaccination of mice which did not get GK1.5 (No GK1.5) and unvaccinated (UnVac) mice; mice were followed for 70 days (10 weeks) after challenge, with percent survival recorded daily; data are from two experiments with a total of 10 mice/group; significant (P<0.001, Mantel-Cox log rank test) survival compared with unvaccinated mice was seen only for mice which did not get GK1.5). Regardless of when the GK1.5 was given, vaccine-mediated protection was lost.

A high percentage of persons infected with HIV have CD4⁺ T cell counts above 100 when they are found to be HIV-infected or following initiation of antiretroviral therapy. To model whether a vaccine would protect this population, we studied doses of GK1.5 that result in only partial depletion of CD4⁺ T cells. In an experiment (n=2 mice/group), we titrated the dose of the anti-CD4 Ab GK1.5 by administering an IP injection and then measuring CD4⁺ counts in the peripheral blood 2 days later. Following injections of 0, 3, 6, 12, 25, 50, 100, and 200 μg of GK1.5, mean CD4⁺ T cell counts were 804, 179, 185, 44, 16, 13, 9, and 9 per μL blood, respectively. Based on this dose-finding experiment, we then injected vaccinated mice with a range of doses of GK1.5 predicted to give varying degrees of CD4⁺ T cell depletion. Vaccine-mediated protection was inversely proportional to the GK1.5 dose (FIG. 14; BALB/c mice were vaccinated thrice at biweekly intervals with GP-Cda1 and GP-Cda2; thirteen days after the last boost, mice were injected IP with the indicated amount of anti-CD4 mAb GK1.5; one day later, the mice received a pulmonary challenge with 2×10⁴ CFU of Cn KN99α; UnVac: Unvaccinated; data are from two independent experiments, each with 5 mice/group), but importantly, even with doses predicted to deplete 98% of the CD4⁺ T cells at the time of challenge, partial protection was maintained. Following a single injection of GK1.5, CD4⁺ T cell counts begin to recover after two to three weeks but do not fully recover for over 10 weeks.

### Example 4: Role of B Cells and Antibody in Vaccine-Mediated Protection

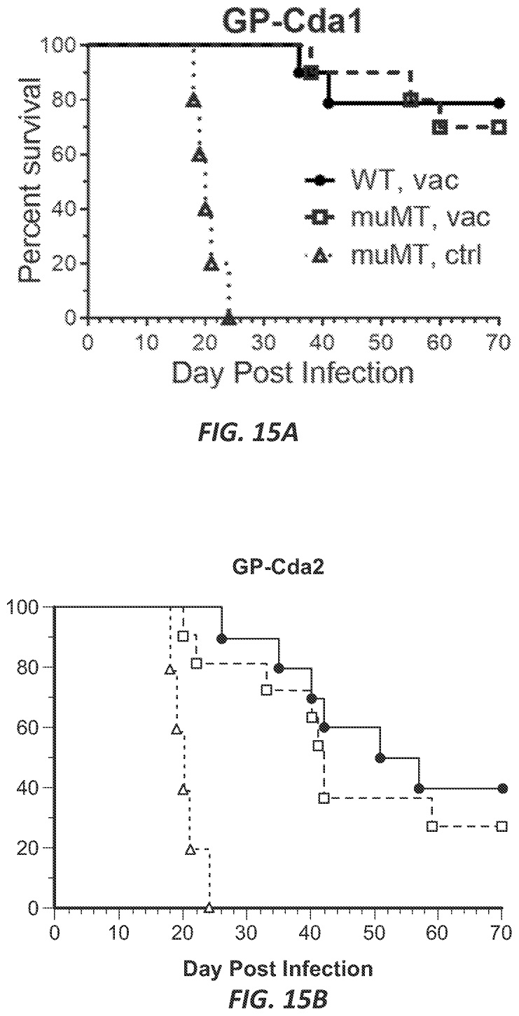

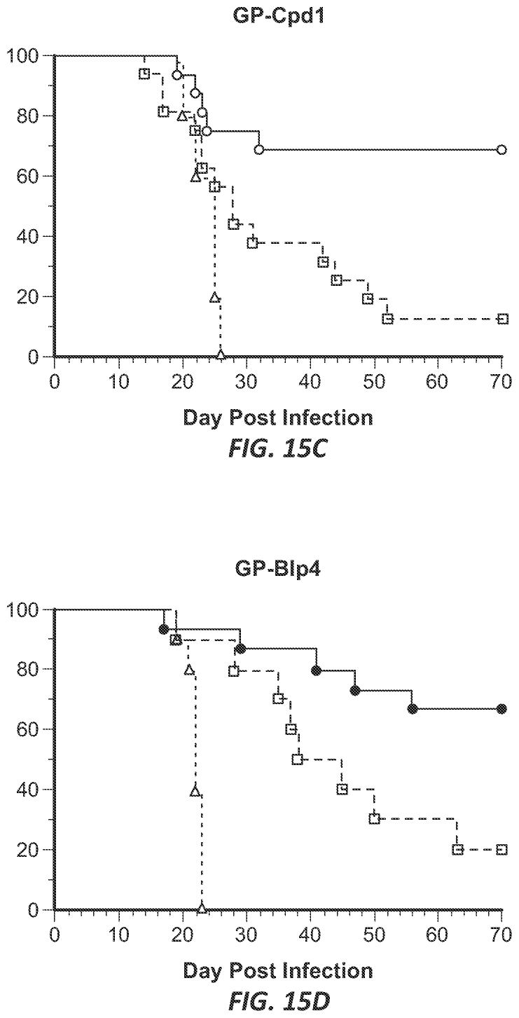

In our preliminary studies, vaccine-mediated protection was lost in CD4-null mice and WT mice fully depleted of CD4⁺ T cells. However, these findings do not preclude a role for B cells in protection. For example, T follicular helper (Tfh) cells provide help to germinal center B cells during affinity maturation. From the standpoint of cryptococcal vaccines, this suggests a rationale for immunizing persons when their CD4⁺ T cell function is relatively intact (such as HIV+ with CD4⁺ counts above 200 or those awaiting solid organ transplant). Therefore, we vaccinated mice congenitally deficient in B cells (μMT, on a C57BL/6 background) with four of our lead candidate vaccines: GP-Cda1, GP-Cda2, GP-Cpd1 and GP-Blp4. Following challenge with C. neoformans, we found μMT mice vaccinated with GP-Cpd1 and GP-Blp4 had significantly increased mortality compared with control mice. However, mortality in the mouse strains was similar following vaccination with GP-Cda1 and GP-Cda2 (FIG. 15). FIG. 15A is with GP-Cda1, FIG. 15B is with GP-Cda2, FIG. 15C is with GP-Cpd1, and FIG. 15D is with Gp-Blp4. Wild-type (WT) C57BL/6 mice and B cell deficient (μMT) mice on the C57BL/6 background received a prime and two biweekly boosts with the indicated subcu GP-based vaccine. One week after the last boost, mice were challenged with 1×10⁴ Cn KN99α and followed for 70 d for survival. Vac=vaccinated. Ctrl=μMT mice receiving control immunizations consisting of GPs with mouse serum albumin. n=10-16 vaccinated mice/group. n=5 control mice/group. P=0.01 and 0.02 comparing WT and μMT mice for GP-Cpd1 and GP-Blp4, respectively (Mantel-Cox log rank test). These data suggest antibodies contribute to protection mediated by GP-Cpd1 and GP-Blp4, but not Cda1 and Cda2.

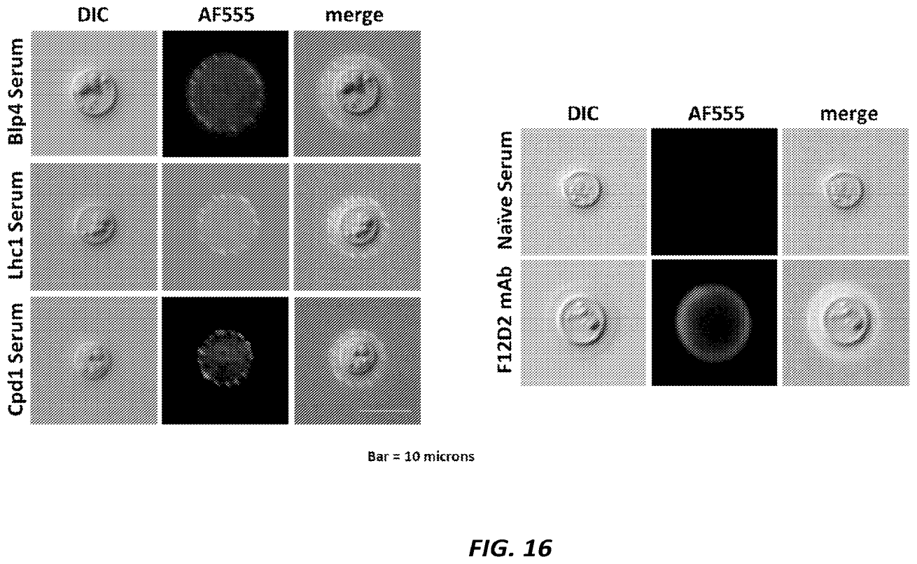

To further test this, in FIG. 16, serum was collected from immunized mice and incubated with C. neoformans. The fungi were then washed, and immunofluorescent staining (AlexaFluor 555 conjugated mouse IgG secondary antibody) was performed. Serum from unimmunized mice (Naïve serum) served as a negative control. The mouse monoclonal antibody F12D2, which is known to react with the capsule of C. neoformans, served as a positive control. As shown, the data in FIG. 16 lend further support to our hypothesis that vaccination with these three proteins could elicit not only protective T cell responses but also protective antibody responses. Moreover, monoclonal antibodies against these proteins could be therapeutic in patients with cryptococcosis.

We hypothesize that the mechanism of protection is linked to the finding that Cpd1 and Blp4 (as well as Lhc1) are found in association with the cryptococcal capsule. Carboxypeptidases cleave peptide bonds from the C-terminus of proteins, so teleologically, it makes sense that this enzyme would be found on the surface of the organism where it could participate in digestion of proteins to amino acids for subsequent uptake. C. neoformans has abundant Cpd1 activity. Interestingly with regards to Blp4, a member of its protein family, Blp1, was shown to mediate capsule-independent inhibition of phagocytosis. Blp4 contains a Barwin-like domain; Barwin domains have saccharide-binding activity, which could explain why Blp4 is capsule-associated. Lhc1 is attached to the outer capsule of C. neoformans where it facilitates immune evasion and virulence by altering capsule structure.

Antibodies to Cpd1, Blp4, and Lhc1 could play a protective role by promoting opsonization, interfering with capsule formation/function, neutralizing enzymatic activity and/or modulating fungal metabolism. Moreover, if antibodies to these surface proteins are shown to be protective, it would have therapeutic implications.

### Example 5: Stimulation of Peripheral Blood Mononuclear Cells (PBMC) from Humans with Cryptococcosis

Critical to the goal of creating a protective human vaccine is demonstrating results from mouse models translate to humans. We and others have shown lymphoproliferative responses to soluble cryptococcal antigens as well as heat-killed C. neoformans by human PBMCs obtained from relatively immunocompetent subjects who have recovered from cryptococcosis. In contrast, PBMCs from healthy donors respond only to the killed yeast cells. Given this knowledge base, we tested T helper cell (Th) responses of human PBMCs to the selected proteins, including Cda1, Cda2, Cpd1, Blp4, and Lhc1. To identify cryptococcal proteins (antigens) that stimulate T cell responses during natural human cryptococcosis, PBMCs from human subjects who have recovered or are recovering from infection were studied. The subjects were enrolled in an ongoing NIH clinical study of cryptococcosis patients at the NIH Clinical Center. Donors who have known quantitative or qualitative disorders of T cell function, such as HIV infection, idiopathic CD4⁺ T cell lymphocytopenia, and lymphoma were excluded. All patients had whole exome sequencing and their HLA types are known.

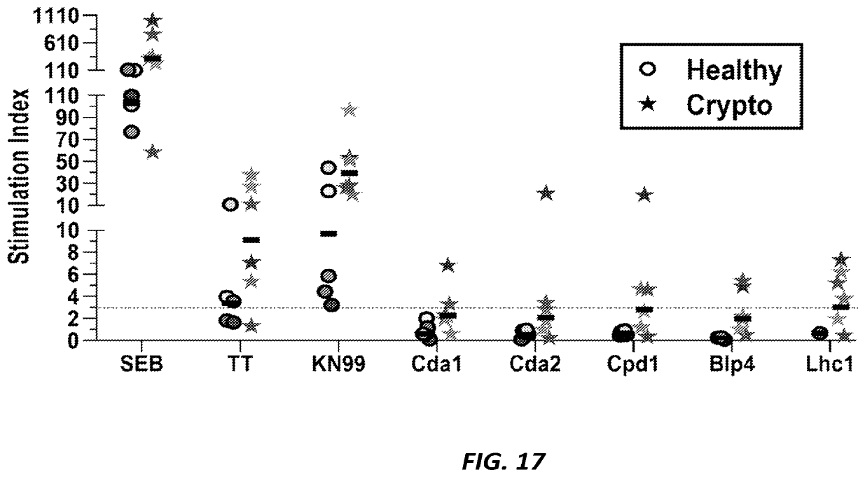

In preliminary studies, PBMCs (stored in liquid N2) from subjects with a recent history of cryptococcosis were obtained and compared to similarly stored PBMCs from healthy blood donors. To measure antigen-specific proliferation/activation/, PBMCs were cultured with antigens. Th cells were then analyzed by flow cytometry for dual expression of Ki67 and PD-1. In studies with healthy donors, we determined that the activation markers Ki67 and PD-1 had high sensitivity and specificity for CD4+ and CD8⁺ T cells. For the five healthy blood donors studied, all had CD4⁺ responses to the heat-killed C. neoformans strain KN99α (FIG. 17; human PBMCs (10⁵/well) from five healthy donors (Healthy) and six donors with cryptococcosis (Crypto) were cultured with the indicated Ags for 7 days in RPM11640/5% human AB serum; each donor is represented by a separate symbol (circle for healthy subjects and star for subjects with cryptococcosis); the media was changed, and cells were restimulated on d3; cells were analyzed by flow cytometry, gating on singlets, live cells, CD3, CD4, Ki67 and PD-1; samples were run in duplicate, and values averaged; data are expressed as Stimulation Index, calculated as sample/unstimulated positive cells, with a ratio >3 (dotted line) considered significant; SEB, staphylococcal enterotoxin B. TT, tetanus toxoid. KN99, heat-killed Cn KN99α) but none responded to the recombinant antigens. Controls were the superantigen, SEB, and tetanus toxoid. As expected, PBMCs from the six cryptococcosis subjects had heterogeneous responses. The striking finding was four of the subjects had a stimulation index (SI) >3 in response to some of the recombinant antigens; all responded to heat-killed C. neoformans strain KN99α. These data suggest that the antigens we are studying are immunogenic in humans infected with Cryptococcus.

### REFERENCES FOR EXAMPLE 3

1 Heitman, J., Kozel, T. R., Kwon-Chung, K. J., Perfect, J. R. & Casadevall, A. 620 (ASM Press, Washington DC, 2010, 2011).
2 Brown, G. D. et al. Hidden killers: human fungal infections. Sci Transl Med 4, 165rv113, doi:10.1126/scitranslmed.3004404 (2012).
3 Jesus, M. D. et al. Glucuronoxylomannan, galactoxylomannan, and mannoprotein occupy spatially separate and discrete regions in the capsule of Cryptococcus neoformans. Virulence 1, 500-508, doi:10.4161/viru.1.6.13451 (2010).
4 Rajasingham, R. et al. Global burden of disease of HIV-associated cryptococcal meningitis: an updated analysis. The Lancet infectious diseases 17, 873-881, doi:10.1016/s1473-3099(17)30243-8 (2017).
5 Singh, N., Dromer, F., Perfect, J. R. & Lortholary, O. Cryptococcosis in solid organ transplant recipients: current state of the science. Clin Infect Dis 47, 1321-1327 (2008).
6 Byrnes, E. J., 3rd et al. Emergence and pathogenicity of highly virulent Cryptococcus gattii genotypes in the northwest United States. PLoS Pathog 6, e1000850 (2010).
7 Byrnes, E. J., 3rd et al. A diverse population of Cryptococcus gattii molecular type VGIII in southern Californian HIV/AIDS patients. PLoS pathogens 7, e1002205, doi:10.1371/journal.ppat.1002205 (2011).
8 Benedict, K. & Park, B. J. Invasive fungal infections after natural disasters. Emerg Infect Dis 20, 349-355 (2014).
9 Greer, A., Ng, V. & Fisman, D. Climate change and infectious diseases in North America: the road ahead. Canadian Medical Association Journal 178, 715-722, doi:10.1503/cmaj.081325 (2008).
10 Wozniak, K. L. & Levitz, S. M. in Cryptococcus: From Human Pathogen to Model Yeast (eds J. Heitman et al.) 387-396 (ASM Press, 2011).
11 Hardison, S. E., Wozniak, K. L., Kolls, J. K. & Wormley, F. L., Jr. Interleukin-17 is not required for classical macrophage activation in a pulmonary mouse model of Cryptococcus neoformans infection. Infection and immunity 78, 5341-5351, doi:10.1128/IAI.00845-10 (2010).
12 Rohatgi, S. et al. Fc Gamma Receptor 3A Polymorphism and Risk for HIV-Associated Cryptococcal Disease. mBio 4, e00573-00513, doi:10.1128/mBio.00573-13 (2013).
13 Chen, L.-C., Goldman, D. L., Doering, T. L., Pirofski, L.-a. & Casadevall, A. Antibody Response to Cryptococcus neoformans Proteins in Rodents and Humans. Infection and immunity 67, 2218-2224 (1999).
14 Fleuridor, R., Lyles, R. H. & Pirofski, L. Quantitative and qualitative differences in the serum antibody profiles of human immunodeficiency virus-infected persons with and without Cryptococcus neoformans meningitis. J Infect Dis 180, 1526-1535 (1999).
15 niaid.nih.gov/topics/malaria/documents/jordan20_2002.pdf. The Jordon Report 20th Anniversary: Accelerated Development of Vaccines 2002. (U.S Department of Health and Human Services, 2002).
16 Molloy, S. F. et al. Antifungal Combinations for Treatment of Cryptococcal Meningitis in Africa. N Engl J Med 378, 1004-1017, doi:10.1056/NEJMoa1710922 (2018).
17 Wang, Y., Wang, K., Masso-Silva, J. A., Rivera, A. & Xue, C. A Heat-Killed Cryptococcus Mutant Strain Induces Host Protection against Multiple Invasive Mycoses in a Murine Vaccine Model. mBio 10, 02145, doi:10.1128/mBio.02145-19 (2019).
18 Colombo, A. C. et al. Cryptococcus neoformans Glucuronoxylomannan and Sterylglucoside Are Required for Host Protection in an Animal Vaccination Model. mBio 10, e02909-02918, doi:10.1128/mBio.02909-18 (2019).
19 Zhai, B. et al. Development of Protective Inflammation and Cell-Mediated Immunity against Cryptococcus neoformans after Exposure to Hyphal Mutants. mBio 6, e01433-01415, doi:10.1128/mBio.01433-15 (2015).
20 Upadhya, R. et al. Induction of Protective Immunity to Cryptococcal Infection in Mice by a Heat-Killed, Chitosan-Deficient Strain of Cryptococcus neoformans. mBio 7, e00547-00516, doi:10.1128/mBio.00547-16 (2016).
21 Ueno, K., Yanagihara, N., Shimizu, K. & Miyazaki, Y. Vaccines and Protective Immune Memory against Cryptococcosis. Biol Pharm Bull 43, 230-239, doi:10.1248/bpb.b19-00841 (2020).
22 Wormley, F. L., Jr., Perfect, J. R., Steele, C. & Cox, G. M. Protection against cryptococcosis by using a murine gamma interferon-producing Cryptococcus neoformans strain. Infection and immunity 75, 1453-1462, doi:10.1128/IAI.00274-06 (2007).
23 Van Dyke, M. C. C. et al. Induction of Broad-Spectrum Protective Immunity against Disparate Cryptococcus Serotypes. Frontiers in Immunology 8, 1-16, doi:10.3389/fimmu.2017.01359 (2017).
24 Levitz, S. M. & Golenbock, D. T. Beyond empiricism: informing vaccine development through innate immunity research. Cell 148, 1284-1292, doi:10.1016/j.cell.2012.02.012 (2012).
25 Murphy, J. W. et al. Serological, electrophoretic, and biological properties of Cryptococcus neoformans antigens. Infect Immun 56, 424-431 (1988).
26 Levitz, S. M. & North, E. A. Lymphoproliferation and cytokines profiles in human peripheral blood mononuclear cells stimulated by Cryptococcus neoformans. J Med Vet Mycol 35, 229-236 (1997).
27 Mansour, M. K., Latz, E. & Levitz, S. M. Cryptococcus neoformans glycoantigens are captured by multiple lectin receptors and presented by dendritic cells. J Immunol 176, 3053-3061, doi:10.4049/jimmunol.176.5.3053 (2006).
28 Mansour, M. K., Schlesinger, L. S. & Levitz, S. M. Optimal T-cell responses to Cryptococcus neoformans mannoprotein are dependent on recognition of conjugated carbohydrates by mannose receptors. J Immunol 168, 2872-2879. (2002).
29 Levitz, S. M. & Specht, C. A. The molecular basis for the immunogenicity of Cryptococcus neoformans mannoproteins. FEMS Yeast Res 6, 513-524, doi:10.1111/j.1567-1364.2006.00071.x (2006).
30 Specht, C. A., Nong, S., Dan, J. M., Lee, C. K. & Levitz, S. M. Contribution of glycosylation to T cell responses stimulated by recombinant Cryptococcus neoformans mannoprotein. J Infect Dis 196, 796-800, doi:10.1086/520536 (2007).
31 Mansour, M. K., Yauch, L. E., Rottman, J. B. & Levitz, S. M. Protective efficacy of antigenic fractions in mouse models of cryptococcosis. Infect Immun 72, 1746-1754, doi:10.1128/iai.72.3.1746-1754.2004 (2004).
32 Mansour, M. K., Yauch, L. E., Rottman, J. B. & Levitz, S. M. Protective efficacy of antigenic fractions in mouse models of cryptococcosis. Infect Immun 72, 1746-1754 (2004).
33 Hester, M. M. et al. Protection of mice against experimental cryptococcosis using glucan particle-based vaccines containing novel recombinant antigens. Vaccine 38, 620-626, doi:10.1016/j.vaccine.2019.10.051 (2020).
34 Specht, C. A. et al. Vaccination with Recombinant Cryptococcus Proteins in Glucan Particles Protects Mice against Cryptococcosis in a Manner Dependent upon Mouse Strain and Cryptococcal Species. MBio 8, e01872-01817, doi:10.1128/mBio.01872-17 (2017).
35 Huang, H. et al. Distinct patterns of dendritic cell cytokine release stimulated by fungal beta-glucans and toll-like receptor agonists. Infect Immun 77, 1774-1781, doi:10.1128/IAI.00086-09 (2009).
36 Osorio, F. & Reis e Sousa, C. Myeloid C-type Lectin Receptors in Pathogen Recognition and Host Defense. Immunity 34, 651-664 (2011).
37 Agarwal, S. et al. Linkage Specificity and Role of Properdin in Activation of the Alternative Complement Pathway by Fungal Glycans. mBio 2, doi:10.1128/mBio.00178-11 (2011).
38 Huang, H. et al. Relative contributions of dectin-1 and complement to immune responses to particulate beta-glucans. J Immunol 189, 312-317, doi:10.4049/jimmunol.1200603 (2012).
39 Specht, C. A. et al. Protection against Experimental Cryptococcosis following Vaccination with Glucan Particles Containing Cryptococcus Alkaline Extracts. mBio 6, e01905-01915, doi:10.1128/mBio.01905-15 (2015).
40 Deepe, G. S., Jr. et al. Vaccination with an alkaline extract of Histoplasma capsulatum packaged in glucan particles confers protective immunity in mice. Vaccine 36, 3359-3367, doi:10.1016/j.vaccine.2018.04.047 (2018).
41 Cole, G. T. et al. Novel strategies to enhance vaccine immunity against coccidioidomycosis. PLoS Pathog 9, e1003768, doi:10.1371/journal.ppat.1003768 (2013).
42 Novak, M. & Vetvicka, V. Beta-glucans, history, and the present: immunomodulatory aspects and mechanisms of action. J Immunotoxicol 5, 47-57 (2008).
43 Weitberg, A. B. A phase I/II trial of beta-(1,3)/(1,6) D-glucan in the treatment of patients with advanced malignancies receiving chemotherapy. J Exp Clin Cancer Res 27, 40 (2008).
44 Williams, D. L. et al. Pre-clinical safety evaluation of soluble glucan. Int J Immunopharmacol 10, 405-414 (1988).
45 Ardiani, A., Higgins, J. P. & Hodge, J. W. Vaccines based on whole recombinant Saccharomyces cerevisiae cells. FEMS yeast research 10, 1060-1069, doi:10.1111/j.1567-1364.2010.00665.x (2010).
46 Schiff, E. R., Everson, G. T. & Tsai, N. HCV-specific cellular immunity, RNA reductions, and normalization of ALT in chronic HCV subjects after treatment with GI-5005, a yeastbased immunotherapy targeting NS3 and core: a randomized, double-blind, placebo controlled phase 1b study [abstract 1304]. Hepatology international 46 (Suppl S1), 816A (2007).
47 Lu, Y. et al. Mutation-selective tumor remission with Ras-targeted, whole yeast-based immunotherapy. Cancer research 64, 5084-5088, doi:10.1158/0008-5472.CAN-04-1487 (2004).
48 Stubbs, A. C. et al. Whole recombinant yeast vaccine activates dendritic cells and elicits protective cell-mediated immunity. Nat Med 7, 625-629 (2001).
49 Wansley, E. K. et al. Vaccination with a Recombinant Saccharomyces cerevisiae Expressing a Tumor Antigen Breaks Immune Tolerance and Elicits Therapeutic Antitumor Responses. Clinical Cancer Research 14, 4316-4325, doi:10.1158/1078-0432.ccr-08-0393 (2008).
50 Christensen, D., Mortensen, R., Rosenkrands, I., Dietrich, J. & Andersen, P. Vaccine-induced Th17 cells are established as resident memory cells in the lung and promote local IgA responses. Mucosal Immunol 10, 260-270, doi:10.1038/mi.2016.28 (2017).
51 Lindenstrom, T. et al. Tuberculosis subunit vaccination provides long-term protective immunity characterized by multifunctional CD4 memory T cells. J Immunol 182, 8047-8055, doi:10.4049/jimmunol.0801592 (2009).
52 Lindenstrom, T. et al. Vaccine-Induced Th17 Cells Are Maintained Long-Term Postvaccination as a Distinct and Phenotypically Stable Memory Subset. Infection and Immunity 80, 3533-3544, doi:10.1128/iai.00550-12 (2012).
53 Schick, J. et al. Cutting Edge: TNF Is Essential for Mycobacteria-Induced MINCLE Expression, Macrophage Activation, and Th17 Adjuvanticity. The Journal of Immunology 205, 323-328, doi:10.4049/jimmunol.2000420 (2020).
54 Pedersen, G. K., Andersen, P. & Christensen, D. Immunocorrelates of CAF family adjuvants. Semin Immunol 39, 4-13, doi:10.1016/j.smim.2018.10.003 (2018).
55 Desel, C. et al. The Mincle-activating adjuvant TDB induces MyD88-dependent Th1 and Th17 responses through IL-1R signaling. PLoS One 8, e53531, doi:10.1371/journal.pone.0053531 (2013).
56 Abraham, S. et al. Safety and immunogenicity of the chlamydia vaccine candidate CTH522 adjuvanted with CAF01 liposomes or aluminium hydroxide: a first-in-human, randomised, double-blind, placebo-controlled, phase 1 trial. Lancet Infect Dis 19, 1091-1100, doi:10.1016/s1473-3099(19)30279-8 (2019).
57 van Dissel, J. T. et al. A novel liposomal adjuvant system, CAF01, promotes long-lived Mycobacterium tuberculosis-specific T-cell responses in human. Vaccine 32, 7098-7107, doi:10.1016/j.vaccine.2014.10.036 (2014).
58 Akilimali, N. A. et al. Cryptococcosis-Associated Immune Reconstitution Inflammatory Syndrome Is Associated With Dysregulation of IL-7/IL-7 Receptor Signaling Pathway in T Cells and Monocyte Activation. J Acquir Immune Defic Syndr 80, 596-604, doi:10.1097/QAI.0000000000001946 (2019).
59 Teitz-Tennenbaum, S. et al. Autocrine IL-10 Signaling Promotes Dendritic Cell Type-2 Activation and Persistence of Murine Cryptococcal Lung Infection. J Immunol 201, 2004-2015, doi:10.4049/jimmunol.1800070 (2018).
60 Panackal, A. A. et al. Paradoxical Immune Responses in Non-HIV Cryptococcal Meningitis. PLoS Pathog 11, e1004884, doi:10.1371/journal.ppat.1004884 (2015).
61 Wiesner, D. L. et al. Chitin recognition via chitotriosidase promotes pathologic type-2 helper T cell responses to cryptococcal infection. PLoS Pathog 11, e1004701, doi:10.1371/journal.ppat.1004701 (2015).
62 Chang, C. C. et al. Cryptococcosis-IRIS is associated with lower Cryptococcus-specific IFN-gamma responses before antiretroviral therapy but not higher T-cell responses during therapy. J Infect Dis 208, 898-906, doi:10.1093/infdis/jit271 (2013).
63 Jarvis, J. N. et al. The phenotype of the Cryptococcus-specific CD4+ memory T-cell response is associated with disease severity and outcome in HIV-associated cryptococcal meningitis. J Infect Dis 207, 1817-1828, doi:10.1093/infdis/jit099 (2013).
64 Chen, Y. et al. The Cryptococcus neoformans transcriptome at the site of human meningitis. mBio 5, e01087-01013, doi:10.1128/mBio.01087-13 (2014).
65 Bremel, R. D. & Homan, J. Extensive T-cell epitope repertoire sharing among human proteome, gastrointestinal microbiome, and pathogenic bacteria: Implications for the definition of self. Frontiers in immunology 6, doi:10.3389/fimmu.2015.00538 (2015).
66 Yauch, L. E., Lam, J. S. & Levitz, S. M. Direct inhibition of T-cell responses by the Cryptococcus capsular polysaccharide glucuronoxylomannan. PLoS pathogens 2, e120, doi:10.1371/journal.ppat.0020120 (2006).
67 Datta, K., Lees, A. & Pirofski, L.-a. Therapeutic Efficacy of a Conjugate Vaccine Containing a Peptide Mimotope of Cryptococcal Capsular Polysaccharide Glucuronoxylomannan. Clinical and Vaccine Immunology 15, 1176-1187, doi:10.1128/cvi.00130-08 (2008).
68 Oscarson, S., Alpe, M., Svahnberg, P., Nakouzi, A. & Casadevall, A. Synthesis and immunological studies of glycoconjugates of Cryptococcus neoformans capsular glucuronoxylomannan oligosaccharide structures. Vaccine 23, 3961-3972, doi:10.1016/j.vaccine.2005.02.029 (2005).
69 Drummond, R. A., Franco, L. M. & Lionakis, M. S. Human CARD9: A Critical Molecule of Fungal Immune Surveillance. Frontiers in Immunology 9, doi:10.3389/fimmu.2018.01836 (2018).
70 Luberto, C. et al. Identification of Appl as a regulator of phagocytosis and virulence of Cryptococcus neoformans. J Clin Invest 112, 1080-1094 (2003).
71 Park, Y. D. et al. A role for LHC1 in higher order structure and complement binding of the Cryptococcus neoformans capsule. PLoS Pathog 10, e1004037, doi:10.1371/journal.ppat.1004037 (2014).
72 Wiesner, D. L. et al. Chitin recognition via chitotriosidase promotes pathologic type-2 helper T cell responses to cryptococcal infection. PLoS pathogens 11, e1004701, doi:10.1371/journal.ppat.1004701 (2015).
73 Levitz, S. M., Nong, S., Mansour, M. K., Huang, C. & Specht, C. A. Molecular characterization of a mannoprotein with homology to chitin deacetylases that stimulates T cell responses to Cryptococcus neoformans. Proc Natl Acad Sci USA 98, 10422-10427, doi:10.1073/pnas.181331398 (2001).
74 Huang, C., Nong, S. H., Mansour, M. K., Specht, C. A. & Levitz, S. M. Purification and characterization of a second immunoreactive mannoprotein from Cryptococcus neoformans that stimulates T-Cell responses. Infect Immun 70, 5485-5493 (2002).
75 Edwards, J. E., Jr. et al. A Fungal Immunotherapeutic Vaccine (NDV-3A) for Treatment of Recurrent Vulvovaginal Candidiasis-A Phase 2 Randomized, Double-Blind, Placebo-Controlled Trial. Clin Infect Dis 66, 1928-1936, doi:10.1093/cid/ciy185 (2018).
76 Coffman, R. L., Sher, A. & Seder, R. A. Vaccine Adjuvants: Putting Innate Immunity to Work. Immunity 33, 492-503, doi:https://doi.org/10.1016/j.immuni.2010.10.002 (2010).
77 Wegmann, F. et al. The Carbomer-Lecithin Adjuvant Adjuplex Has Potent Immunoactivating Properties and Elicits Protective Adaptive Immunity against Influenza Virus Challenge in Mice. Clinical and vaccine immunology: CVI 22, 1004-1012, doi:10.1128/CVI.00736-14 (2015).
78 Gasper, D. J. et al. Effective Respiratory CD8 T-Cell Immunity to Influenza Virus Induced by Intranasal Carbomer-Lecithin-Adjuvanted Non-replicating Vaccines. PLoS pathogens 12, e1006064-e1006064, doi:10.1371/journal.ppat.1006064 (2016).
79 Cluff, C. W. et al. Synthetic Toll-Like Receptor 4 Agonists Stimulate Innate Resistance to Infectious Challenge. Infection and Immunity 73, 3044-3052, doi:10.1128/iai.73.5.3044-3052.2005 (2005).
80 Tan, Z. Y. et al. Synthetic TLR4 agonist as a potential immunotherapy for melioidosis. Open Journal of Immunology Vol. 03No. 01, 9, doi:10.4236/oji.2013.31001 (2013).
81 Smith, A. J. et al. Species-Specific Structural Requirements of Alpha-Branched Trehalose Diester Mincle Agonists. Frontiers in Immunology 10, doi:10.3389/fimmu.2019.00338 (2019).
82 Desjardins, C. A. et al. Population genomics and the evolution of virulence in the fungal pathogen Cryptococcus neoformans. Genome Res 27, 1207-1219, doi:10.1101/gr.218727.116 (2017).
83 Ashton, P. M. et al. Three phylogenetic groups have driven the recent population expansion of Cryptococcus neoformans. Nature Communications 10, 2035, doi:10.1038/s41467-019-10092-5 (2019).
84 Mukaremera, L. et al. The Mouse Inhalation Model of Cryptococcus neoformans Infection Recapitulates Strain Virulence in Humans and Shows that Closely Related Strains Can Possess Differential Virulence. Infect Immun 87, doi:10.1128/IAI.00046-19 (2019).
85 Wiesner, D. L. et al. Cryptococcal Genotype Influences Immunologic Response and Human Clinical Outcome after Meningitis. mBio 3, e00196-00112, doi:10.1128/mBio.00196-12 (2012).
86 Thanh, L. T. et al. Assessing the virulence of Cryptococcus neoformans causing meningitis in HIV infected and uninfected patients in Vietnam. Med Mycol 58, 1149-1161, doi:10.1093/mmy/myaa013 (2020).
87 Fraser, J. A. et al. Same-sex mating and the origin of the Vancouver Island Cryptococcus gattii outbreak. Nature 437, 1360-1364, doi:10.1038/nature04220 (2005).
88 Armstrong-James, D. et al. Immunotherapeutic approaches to treatment of fungal diseases. Lancet Infect Dis 17, e393-e402, doi:10.1016/S1473-3099(17)30442-5 (2017).
89 Szabo, P. A., Miron, M. & Farber, D. L. Location, location, location: Tissue resident memory T cells in mice and humans. Sci Immunol 4, doi:10.1126/sciimmunol.aas9673 (2019).
90 Hagen, F. et al. Autochthonous and dormant Cryptococcus gattii infections in Europe. Emerg Infect Dis 18, 1618-1624, doi:10.3201/eid1810.120068 (2012).
91 Davis, M. J. et al. Pulmonary Iron Limitation Induced by Exogenous Type I IFN Protects Mice from <span class=“named-content genus-species” id=“named-content-1”>Cryptococcus gattii</span> Independently of T Cells. mBio 10, e00799-00719, doi: 10.1128/mBio.00799-19 (2019).
92 Althoff, K. N. et al. CD4 count at presentation for HIV care in the United States and Canada: Are those over 50 years more likely to have a delayed presentation? AIDS Research and Therapy 7, 45, doi:10.1186/1742-6405-7-45 (2010).
93 Rice, J. C. & Bucy, R. P. Differences in the degree of depletion, rate of recovery, and the preferential elimination of naive CD4+ T cells by anti-CD4 monoclonal antibody (GK1.5) in young and aged mice. J Immunol 154, 6644-6654 (1995).
94 Capilla, J., Maffei, C. M., Clemons, K. V., Sobel, R. A. & Stevens, D. A. Experimental systemic infection with Cryptococcus neoformans var. grubii and Cryptococcus gattii in normal and immunodeficient mice. Med Mycol 44, 601-610, doi:10.1080/13693780600810040 (2006).
95 Nanjappa, S. G., Heninger, E., Wuthrich, M., Gasper, D. J. & Klein, B. S. Tc17 cells mediate vaccine immunity against lethal fungal pneumonia in immune deficient hosts lacking CD4+ T cells. PLoS pathogens 8, e1002771, doi:10.1371/journal.ppat.1002771 (2012).
96 Wuthrich, M., Filutowicz, H. I., Warner, T., Deepe, G. S., Jr. & Klein, B. S. Vaccine immunity to pathogenic fungi overcomes the requirement for CD4 help in exogenous antigen presentation to CD8+ T cells: implications for vaccine development in immune-deficient hosts. J Exp Med 197, 1405-1416, doi:10.1084/jem.20030109 (2003).
97 Lionakis, M. S. & Levitz, S. M. Host Control of Fungal Infections: Lessons from Basic Studies and Human Cohorts. Annu Rev Immunol 36, 157-191, doi:10.1146/annurev-immunol-042617-053318 (2018).
98 Cook, S. L., Franke, M. C., Sievert, E. P. & Sciammas, R. A Synchronous IRF4-Dependent Gene Regulatory Network in B and Helper T Cells Orchestrating the Antibody Response. Trends Immunol, doi:10.1016/j.it.2020.05.001 (2020).
99 Clarke, S. C. et al. Integrated Activity and Genetic Profiling of Secreted Peptidases in Cryptococcus neoformans Reveals an Aspartyl Peptidase Required for Low pH Survival and Virulence. PLoS Pathog 12, e1006051, doi:10.1371/journal.ppat.1006051 (2016).
100 Chun, C. D., Brown, J. C. S. & Madhani, H. D. A Major Role for Capsule-Independent Phagocytosis-Inhibitory Mechanisms in Mammalian Infection by Cryptococcus neoformans. Cell Host & Microbe 9, 243-251, doi:https://doi.org/10.1016/j.chom.2011.02.003 (2011).
101 de Oliveira, A. L. et al. The Structure of the Elicitor Cerato-platanin (CP), the First Member of the CP Fungal Protein Family, Reveals a Double WO-Barrel Fold and Carbohydrate Binding. Journal of Biological Chemistry 286, 17560-17568 (2011).
102 McClelland, E. E., Nicola, A. M., Prados-Rosales, R. & Casadevall, A. Ab binding alters gene expression in Cryptococcus neoformans and directly modulates fungal metabolism. The Journal of Clinical Investigation 120, 1355-1361, doi:10.1172/JC138322 (2010).
103 Kozel, T. R., Tabuni, A., Young, B. J. & Levitz, S. M. Influence of opsonization conditions on C3 deposition and phagocyte binding of large- and small-capsule Cryptococcus neoformans cells. Infect Immun 64, 2336-2338 (1996).
104 Levitz, S. M. & Tabuni, A. Binding of Cryptococcus neoformans by human cultured macrophages. Requirements for multiple complement receptors and actin. J Clin Invest 87, 528-535, doi:10.1172/JCI115027 (1991).
105 Shao, W. et al. Identification of Liver Epithelial Cell-derived Ig Expression in μ chain-deficient mice. Scientific Reports 6, 23669, doi:10.1038/srep23669 (2016).
106 Dromer, F., Perronne, C., Barge, J., Vilde, J. L. & Yeni, P. Role of IgG and complement component C5 in the initial course of experimental cryptococcosis. Clinical & Experimental Immunology 78, 412-417 (1989).
107 Levitz, S. M. & North, E. A. Lymphoproliferation and cytokine profiles in human peripheral blood mononuclear cells stimulated by Cryptococcus neoformans. J Med Vet Mycol 35, 229-236 (1997).
108 Panackal, A. A. et al. Paradoxical Immune Responses in Non-HIV Cryptococcal Meningitis. PLoS pathogens 11, e1004884, doi:10.1371/journal.ppat.1004884 (2015).
109 Miller, G. P. G. & Puck, J. In vitro human lymphocyte responses to Cryptococcus neoformans. Evidence for primary and secondary responses in normals and infected subjects. Journal of Immunology 133, 166-172 (1984).
110 http://clinicaltrials.gov/ct2/show/NCT00001352. Evaluation and Follow-up of Patients with Cryptococcosis, 2014).
111 Chang, C. C. et al. Cryptococcosis-IRIS is associated with lower Cryptococcus-specific IFN-gamma responses before antiretroviral therapy but not higher T-cell responses during therapy. J Infect Dis 208, 898-906, doi:10.1093/infdis/jit271 (2013).
112 Huang, H., Ostroff, G. R., Lee, C. K., Specht, C. A. & Levitz, S. M. Characterization and optimization of the glucan particle-based vaccine platform. Clin Vaccine Immunol 20, 1585-1591, doi:10.1128/CVI.00463-13 (2013).
113 Guerra, E. S. et al. Central Role of IL-23 and IL-17 Producing Eosinophils as Immunomodulatory Effector Cells in Acute Pulmonary Aspergillosis and Allergic Asthma. PLoS Pathog 13, e1006175, doi:10.1371/journal.ppat.1006175 (2017).
114 Reiss, S. et al. Comparative analysis of activation induced marker (AIM) assays for sensitive identification of antigen-specific CD4 T cells. PLoS One 12, e0186998, doi:10.1371/journal.pone.0186998 (2017).
115 Teng, F., Meng, X., Kong, L. & Yu, J. Progress and challenges of predictive biomarkers of anti PD-1/PD-L1 immunotherapy: A systematic review. Cancer Lett 414, 166-173, doi:10.1016/j.canlet.2017.11.014 (2018).
116 Wang, S.-R. et al. OMIP 071: A 31-Parameter Flow Cytometry Panel for In-Depth Immunophenotyping of Human T-Cell Subsets Using Surface Markers. Cytometry Part A 99, 273-277, doi:https://doi.org/10.1002/cyto.a.24272 (2021).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A vaccine for cryptococcosis, comprising: 1) an antigenic peptide or an antigenic protein and 2) an adjuvant, wherein the antigenic peptide or antigenic protein is derived from a protein listed in Table 1.

2. The vaccine of claim 1 comprising an antigenic peptide, wherein the antigenic peptide comprises a sequence that is at least 80% identical to an amino acid sequence of any one of SEQ ID NOs: 1-11.

3. The vaccine of claim 2, wherein the antigenic peptide has an amino acid sequence of SEQ ID NO: 1.

4. The vaccine of claim 2, wherein the antigenic peptide has an amino acid sequence of SEQ ID NO: 9.

5. The vaccine of claim 2, wherein the antigenic peptide has an amino acid sequence of SEQ ID NO: 10.

6. The vaccine of claim 2, wherein the antigenic peptide has an amino acid sequence of SEQ ID NO: 11.

7. The vaccine of claim 2, wherein the adjuvant is either glucan particles or CAF01.

8. The vaccine of claim 1 comprising an antigenic protein, wherein the antigenic protein has an amino acid sequence that is at least 80% identical to an amino acid sequence of any one of SEQ ID NOs: 12-33.

9. The vaccine of claim 8, wherein the antigenic protein has an amino acid sequence that is at least 80% identical to an amino acid sequence of any one of SEQ ID NOs: 12-21.

10. The vaccine of claim 9, wherein the adjuvant is CAF01.

11. The vaccine of claim 8, wherein the antigenic protein has an amino acid sequence that is at least 80% identical to an amino acid sequence of any one of SEQ ID NOs: 22-33.

12. The vaccine of claim 11, wherein the adjuvant is glucan particles or CAF01.

13. A method of protecting a subject against cryptococcosis infection, the method comprising: administering to a subject in need thereof the vaccine of claim 1 for protection against, or treatment of cryptococcosis infection in the subject.

14. A method of protecting a subject against cryptococcosis infection, the method comprising: 1) generating an antibody using a composition comprising a) an antigenic protein or antigenic peptide derived from a protein listed in Table 1 and b) an adjuvant and 2) administering the generated antibody to the subject in need thereof for protection against, or treatment of cryptococcosis infection in the subject.

15. The method of claim 14, wherein the antigenic peptide has an amino acid sequence of any one of SEQ ID NOs: 1-11.

16. The method of claim 14, wherein the antigenic protein has an amino acid sequence of any one of SEQ ID NOs: 12-33.

17. The method of claim 13, wherein the subject is a human or a koala.

18. The method of claim 14, wherein the subject is a human or a koala.


SEQUENCE LISTING (33 sequences)

SEQ ID NO: 1 - protein, 32 aa, Synthetic construct
RAHDEGHEICVHTWSHQYMTALSNEVVFAELY

SEQ ID NO: 2 - protein, 31 aa, Synthetic construct
ENNQKATMFFIGSNVLDWPLQAMRAHDEGHE

SEQ ID NO: 3 - protein, 32 aa, Synthetic construct
TWGLGFDDGPNCSHNALYDLLLENNQKATMFF

SEQ ID NO: 4 - protein, 34 aa, Synthetic construct
EVVFAELYYTQKAIKAVLGVTPQCWRPPYGDVDN

SEQ ID NO: 5 - protein, 32 aa, Synthetic construct
TAYSYAPVTELISSFPTIWQTASIPSNDTEAQ

SEQ ID NO: 6 - protein, 34 aa, Synthetic construct
MSVFMTMFPKIKSAFNYIVPICTAYNITQPYAES

SEQ ID NO: 7 - protein, 35 aa, Synthetic construct
DKAGNGTYTTHGPVVLNHELTNYTMSVFMTMFPKI

SEQ ID NO: 8 - protein, 35 aa, Synthetic construct
KINSTLNTKIPNDVPHGTPTGDWTGVNYSNSDPDC

SEQ ID NO: 9-11 - skipped (3 entries)

SEQ ID NO: 12 - protein, 470 aa, Cryptococcus neoformans
MFTFAAFSALLISLAGVVAQTTGTSVDSSILTKTADSTGPSGFSIPALSELTSGAPTDSTVALYSTFAAGATPTVSGAPVLPTSALTIADYPALDVTPPTNSSLVKDWMAKIDLSKVPSYNVTTGDCSTDAAAISDGRCWWTCGGCTRETDIVECPDKNVWGLSYDDGPSPFTPLLIDYLQEKNIKTTFFVVGSRVLSRPEMLQTEYMSGHQISIHTWSHPALTTLTNEEIVAELGWTMKVIKDTLGVTPNTFRPPYGDIDDRVRAIAAQMGLTPVIWTSYTDGSTTVNFDTNDWHISGGTATGASSYETFEKILTEYAPKLDTGFITLEHDIYQQSVDLAVGYILPQVLANGTYQLKSIINCLGKDTSEAYIETSSNQTTTQITAATGSQSTFFQPIVGTATGAEVSAPSEATGSTAAGSAASTTSGSGASASTGAASNTSSSGSGRSATMGGALIALAAVAVGMVYVA

SEQ ID NO: 13 - protein, 466 aa, Cryptococcus sp.
MSAFAAVSALLVSLAGIMAQTASTSVASSVLTQTAPTGPSGFSIPALSELTSGAPTDTTVALYSTFPASATPTVSGAPVLPTSALTIANYPALDVIPPTNSSLVQEWLAKIDMTKVPNYNATTGDCSTDPGATSDGRCWWTCGGCTRATDIVACPDKNVWGLSYDDGPSPFTPLLIDYLQEKNIKTTFFVVGSRVLSRPEMLQTEYMSGHEISIHTWSHPALTTLTNEQIVAELAWTMKVIKDTIGVTPNTFRPPYGDIDDRVRAIAAQMGLTPVIWTSYSDGSTTVNFDTNDWHISGGTATGASSYETFEKILNEYAPKLNTGFITLEHDLYQQSVDLAVGYILPQVLANGTYQLKSIINCLGKDISEAYIETSSNQTTTQITSASGSTYFQPIVGTATGSEVSAPSEATGSTAAGSAAGSAATTSGSAASGASDSSSSGSGRSATMGGALIAFAAVAVGIVYVA

SEQ ID NO: 14 - protein, 455 aa, Cryptococcus neoformans
MIPSTAAALLTLTAGAAFAHTGCGGHEIGRRNVGGPMLYRRAVTDEASAAVSTDINTECTAYSYAPVTELISSFPTIWQTASIPSNDTEAQQLFGKINSTLNTKIPNDVPHGTPTGDWTGVNYSNSDPDCWWTHNKCTTPSNDTGLQADISIAPEPMTWGLGFDDGPNCSHNALYDLLLENNQKATMFFIGSNVLDWPLQAMRAHDEGHEICVHTWSHQYMTALSNEVVFAELYYTQKAIKAVLGVTPQCWRPPYGDVDNRVRMIAEGLNLTTIIWSDDTDDWAAGTNGVTEQDVTNNYQSVIDKAGNGTYTTHGPVVLNHELTNYTMSVFMTMFPKIKSAFNYIVPICTAYNITQPYAESNITCPNFETYISGVTNISSSTTQKDGSSSTNTASGSGAAGSASATSSSDDSSSSGGSSGSSGSNNASSGALGMFDSLSGVGLILGGVVAGVMLL

SEQ ID NO: 15 - protein, 460 aa, Cryptococcus sp.
MIPSTAAAFLTLTAGTAFAHIGCGGQEIGRRNVGGPMLHSRAVTDEASAAVSTDVSTECTAYGYAPVTQIASSFPAIWQTASILSTDSEAQQLFASINATVNSKLPNDVPHGTPTGNWTGVSYSSSDPDCWWTHNKCTTPSSDTGLKADITTVPEPMTWGLGFDDGPNCSHNALYDLLLENNQKATMFYIGSNVMDWPLQAMRAHDEGHEICVHTWSHQYMTALSNEVVFAELYYTQKAIKAVLGVTPLCWRPPYGDVDNRVRMIAAALNLSTIVWSDDTNDWEAGTNGVTQQDVTNNYQSVIDKAGNGTYTTHGPVVLNHELTNYTMSVFVSMFPKIKSAFSYIVPICTAYNITQPYAESNVTCPNFETYISGVTNISTSTTQKDGSSSTNTSYTASGSTSPSASSTGKSDDSSSSGSASSSTAANSASSAKSGALGMYDGLSGMGLILGGVVAGVMLL

SEQ ID NO: 16 - protein, 412 aa, Cryptococcus neoformans
MYGHLSLSALSLFAVVAAAPFRESWLQPRDSPVSQLFRRTAPDPNSNDYMSYYPGPGSTPNVSTIPQAWLDKLATVNLPNVPVATPDGGRPTYPNNEDDGDSTICSFTDQCRVEDDLYSPPGEKIWALSFDDGPTDVSPALYDYLAQNNISSSATHFMIGGNVITSPQSVLVAVKAGGHLAVHTWSHPYMTTLTNEQVVGELGWTMQALSDLNGGRIPMYWRPPYGDVDNRVRAIAKEVFGLVTVLWDSDTNDWAITDEPGQYSVASVEAYFDTLVTGNRTQGLLLLEHELDNNTVEVFETEYPKAVGNGWTVKNVADAFNMEWYLNSGKGNNDVVTTMSVAGTLTTATPTNTSTYVASSTAASSASVTDSAGVSIASAASSEASSSWAIANRPSHFVIAIACGLALAAIMV

SEQ ID NO: 17 - protein, 410 aa, Cryptococcus sp.
MYGHLSLSALSLLAVVAAAPFHESWLQPRDSPVSQLFRRAAPDPNASDYLSHYPSPGSTPNVSTIPQAWLDKLATVQLPNVSVATASGEIPTYPNNENDGDSTICSFTDQCVEPDDLFSPPGEKIWALSFDDGPTDVSPGLYDFLAQNNISSKATHFMIGGNVVTSPQSVLIAVQAGGHLAVHTWSHPYMTTLTNEQVVGELGWTMQALSDLNGGRVPKFWRPPYGDVDNRVRAIAKGVFDLETVLWDEDTNDWAIADEPSQYSIASVEAYFDTLVTGNRTQGLLLLEHELDNNTVTVFETEYPKAIANGWIVKNVADAFNMEWYLNSGKGNDATVTTMSVGGTLPTAAPTNTSTSVASASATSSGSVTDSAGVSIASAASSESSSSWAIAERPSLFIIACGLVFAAAVV

SEQ ID NO: 18 - protein, 249 aa, Cryptococcus neoformans
MKFITTLFAVLAILSSVSASPTMKKRATVETIDNCSQQGTVALTFDDGPYDYEAQVASALDGGKGTFFLNGANYVCIYDKADSIRALYDAGHTLGSHTWSHADLTQLDESGINDELSKVEDAFVKILGVKPRYFRPPYGNINDNVLKVLGERGYTKVFLWSDDTGDANGESVSYSEGVLDGVIQDYPNPHLVLDHSTIETTSSQVLPYAVPKLKSAGYQLVTVGECLGTDESPYEWVDCPGERDSSWQC

SEQ ID NO: 19 - protein, 311 aa, Cryptococcus sp.
MSLDRPYGGVPSSFALPYRIPFAIRLLPFLPLSKEYKCQPSTTSLLFFLQPSSDKEKSYPIKMKFITTFFTVLAILSSASASPTVKKRATVETIDNCSQQGTVALTFDDGPYNYEAQVASALDGGKGTFFLNGANYACIYDKADSIKALYDAGHTLGSHTWSHADLTQLDESGINDELSKVEDAFVKILGVKPLYFRPPYGNINDNVLNVLSQRGYKKVFMWSDDTGDANGESVSYSEGVLDKVIQNYPNPHLVLEHSPIETTSSQVLPYAVPKLKNAGYQLVTVGECLGTNESPYEFVGCPGERDSSWQC

SEQ ID NO: 20 - protein, 321 aa, Cryptococcus neoformans
MFAKAAVIALASASIVAAAPVNCARAKPTTYDEGYLESYDSYHARYLALSCYSQHNTTFFDDCCHPLLATETLADNRASYCTPNSTAVASVNATIAEATASATASADIEAESQYNNASSYAAEATAPVTTSAEATAPVTASAEATASVTAAAVNNVAEVAQQSASASSEEEQPTASSSSSYGRASASSSSSEEESTSTSSSSASDSSSTSSSQVYTGGYATFFSQGGVAGECGTVHSDDDYVIAIDSNGWWQDYESNDSSPYCGKHITLTNTNNGKSVTAVVADVCPTCETANSLDLSIGAFNQIATEEDGMVPITWYFTD

SEQ ID NO: 21 - protein, 303 aa, Cryptococcus sp.
MFAKAAVIALASASIVVAAPVNCARSKPSTYAEGYLEDYDTYHARYLALSCNTQHNTTFFDDCCHPLLANETLADNRASYCTPNSTAVASINATMAEGAAATASATASADVEAESQYNNASSSDATASVTASSEATASETASALNNVAEIAQQSASATSSSEEQPTSASSSASEESAASTSSSSASTSSSTASSQVYTGGYATFYSQDGVAGACGTMHSDSDYVIAIDSNGWWQDYESNDNSPYCGKQITLTNTNNGKSVTATVADVCPTCETNNSLDLSIGAFNQIATEEEGMVPITWYFVN

SEQ ID NO: 22 - protein, 181 aa, Cryptococcus neoformans
MMSSATAELCFDCANCVEMLPKGTTKSVTRPEILERLTRIENHIEVQTALLSTMAASKDYEKKVYTDDDDELEEPEVQLCNMTCEMAALKTYKPQTKVSNHKIVIEAALGRLDHVNGESSIRWAQSVILGLQAAETLQNDKQMMELARVIGCLEACELRWAGDWRAGVASITLKELRTLMI

SEQ ID NO: 23 - protein, 181 aa, Cryptococcus sp.
MMSSAPSELCFDCANCVEMLPKGTKKSATRPEILERLTRIEDRIEIQTTLLRGMATDRDYEKKVYTDDDDELEESEVPVCNITREMAALKTYKAQPKVTNHKVIIEAALGRLDHVNGDNSIRWAQSVILGLQAAETLQNDRQMMELARVIGCLEACELRWAGDWRAGVASITLKELRTLMI

SEQ ID NO: 24 - protein, 479 aa, Cryptococcus neoformans
MATNKNPDPHAKITSREMSVNTAGLQEWGMGGKTLPAPLGSPVKFILGAKVLSWKSKLVYAGFTMGALATLAYTASHILPSSSIAQASHLTTFKRRATPSEPPKNAQVINPKDFTVLPTVLPSYEFNGSSLFVPPGTTEQSLKAKPFHVYDDSFYDVIGSNPTLTLIADSGSDPLFHEAVVWYKATDEVFFVQNAGAKSAGTGLNKSAVVEKISLLEAAAVQKGEKHEAQVTVVNSTVQVVNPNGGTAYRGKIVFAGEGQGANVPPALYMLDPNEPYPTTVILNNYFGRQFNSLNDISVNPRNKELYFTDVTYGYLQDFRPPPGLPNQVYRFNMDTGVITVVADGLNMPNGLTFSPDGRHAYVTDTGIFSGFWGTNYTYPATIYRWDVQDDGTWENQKVFAYIHVGAADGIHTDSNGNVYAGVGDGVHVFNPHGLLIGKIYLGETSANFNFAGNGRMIICAETHLYYATLGASGWDPEA

SEQ ID NO: 25 - protein, 465 aa, Cryptococcus sp.
MSINSADLQEREMEGKTLPAPLGSSMKSIPGAKGFSWKPKFVYMGLMAALATLAYITIYTLPFAVIARAPSHLSTFKRRSGPREPPEYAQRIYPKEFAVLDTVPPPTEFDGSSLFVPPGTTKESLKAKPFHIYDDSFLDIIGTNPTLTLIADSGTNPLFHEAVVWYTVKDEVLFTQNAGAKAAGTGLNKSAVIEKILLSEAAAVQKHERNQTEVFVVNSTVQVMNPNGATPYGDKFIFAGQGQGPNVPPALYMLDPEEPYHTTIILNNFFGRQFNSLNDVSVNPRNGEIYFTDVMYAYLQDFRPAPGLPNQVYRFNLETGLVQVVADGINMPNGITFSPDGRHAYVSDTAIFSAFWGTNYTYPATIYRWDVEDDGTWSNRKLFAYIHVGAADGIHTDSHGNLYAGVGDGIHVFNPSGMLIGKIYLGETSANFNFAGHGRMVICAETHLYYATLGVSGWVPKAKTV

SEQ ID NO: 26 - protein, 416 aa, Cryptococcus neoformans
MFAVAALASLLSAIAVKAVPCVQFDSSWNLYAFGGDQDVMIGDNTTWSSPSTTPLSTSGRPPWTGNNTQCILSQTNNAMYVIGADSDDLSSIYVYDFSGDSWSTQSTSRTPSDLGNSRSSTVLDHDTNVFFTLTIDSGLYQLDLSSIRNSASRDTLRWEAVSNPSFSVDDYTVTAAQAANHIFFFGTPGTAAGSAHIFVVHYAYFQPEAQAFNGTAFPNAAGQAISIPNVDNNVPYSMVFIPDDFSDTYIVTHWTDLSNYSVTSDAPFDSDLINSTQTLPAPTSQDATAAYAASPYALVQVDAAGDIYYMSNPVQSDYTVSSSASWEKLGYSLIGVRESTSNNSSSSSTASSTSTATGAAAGSASGSVTRSSSTGDASASASSASNSSSGARKMATRGDALGLSMGALVLVAGILM

SEQ ID NO: 27 - protein, 411 aa, Cryptococcus sp.
MLAAAALASLLATIAVNAVTCVQFDSSWNLYAFGGDQDVKIGDNNTWSSPSTTPLSTTGRPPWTGNNTQCILSQTNNAMYVIGADSDDLSSIYVYDFAGNSWSTQNTSRTPSDLGNSRSSSVLDHDTNVFFTLTTDSGLYQLDLSSITNSASSDTLRWEAVENPSFSVDGYFVTAAQAANHIFYFGAPGAASGSAYIFVVHYAYFQPKAQAFNGTAFPDASGQAISIPSAANNVPYSMVFIPHDFSDTYIVTHWTDLSDYSVTSDAPFDVNLINSTQTLPAPTSQDKAAAYAASPYAIVQIDAAGDIYYMSSPVQSDYTVSSSASWEKLGYSLTLSKSKDTSSSSTTSGTSTTTGATHGSASGTASRPGSTDNASASASTNSSSGARRMATRGDVLGLFVGALAVAAGILM

SEQ ID NO: 28 - protein, 548 aa, Cryptococcus neoformans
MCSKVVSAVLLAFALGSVIEAAREPHGLRGRGPAALAAKEAKIEALANNASNNDASKVEQCRTPKERHPKWRFYNNKTSEFLIKSLPDVPFDLGEIYSGLIPIDYHNQSEGLFFVFQPKLGEASDDITIWLNGGPGCSSLEGWFQENGLWTWQAGTYAPVINHYSWVNLTNMLWVEQPIGTGFSIGTPKATTEEEIAQDFIKWFKNFQDLFGIKNYKIYVTGESYAGRYVPYIGAAMLDAQNKTYYDLSGALVYDPAIGESVFVQEQITTYPFVEANANLFNFNKTTMAEFKELHETCGYKDYIERYLKFPPTENQPPLFFDYYDIDNVTCAIFDWVIKEAFRINPCFDIYEINLMCPLLWDVLGMPTQFSYAPGGIYFNRSDVKAAIHAPEHIDWTLCATRPVFVGGEEQGPQGRGDLSADPIQKVLPQVIEATNRVLISNGDFDMVIITNGTLLAIQNMTWNGHLGFQSPPSEDIFIDIVDTQWSSIFESNGYHGYPGAQGVMGIQHYERGLMWAQTYQSGHMQPRYQPRSSYRHLQWLLGYVDRL

SEQ ID NO: 29 - protein, 544 aa, Cryptococcus sp.
MWSKVVTVALLAIALGSIIEARDFHNLRGRGSPALATNKEAKKEVLTTRQSKTKDKCHARPERCPEMKRFYNEQTSEFFVESLPDVPFDLGEIYSGLIPIDYGNQSEALFFVFQPKLGECSDDLTIWLNGGPGCSSLIGFFQENGLWTWQPGTYAPVINPYSWVNLTNMLWVEQPIGTGFSIGTPKATTEEEIAQDFIKWFKNFQDIFGIKNYKIYVSGESYAGRYVPYIGAAMLDQQDKTYYDLSGALVYDPAIGETIFVQEQIPTYPFVEANANLFNFDKTTMAELKELHETCGYQDYIDRYLKFPPTENQPHLYYDYFDYDNATCGIFDKVLAKASRINPCFDIYAINQMCPLLWDILGTPTQLDYAPGGIYFNRSDVKASIHAPSHIDWTACATQPVFVGGEDGPQSRGDVSLDPIQKVLPQVIEATNRVLISNGDFDYVILTNGTLLSIQNMTWNGQLGFQSAPNEDIVIDIIDTQWSSVYEANYREGYPGAQGVMGIQHYERGLMWAETFQSGHMQPQFQPRSAYRHLQWLLGHIDKL

SEQ ID NO: 30 - protein, 83 aa, Cryptococcus neoformans
MSDAGRQSFTDKAGAAMKPDSEKSYLEQAKDTIGGKADSAASTGQPQSQKSYTQEIGDAFSGNKNDNQESLTDKAKNAFGANQ

SEQ ID NO: 31 - protein, 83 aa, Cryptococcus sp.
MSDTGRQSLTDKAGAALKPDSEKSYLEQAKDTISGKTDSAASTAQPQSQKSYTQEIGDAFSGNKNDNQESLTDKAKNAFGANQ

SEQ ID NO: 32 - protein, 381 aa, Cryptococcus neoformans
MLTLLAIALPITIALRAGATVYPLVESWHGEGFFDGFTFPVETYDNTTNGDTFWATPANTSLLYTTSSGTTILKVDNSTFVPYLEKRFAPKLLSKSAYDIGTVWVFDAVHLPYGCSVWPAFWTQGPSWPAGGEIDIVEGINLQATNMIALHTSGASSCTIPTTSPSSFSGTVSYPNCDNSQNYGSGCTVYDTNTNSYGREFAEAGGGVYVAEFARDGIRIWFMTRSAIPDAIQVNATQIDTSSLGTPVAEYPSTSCDIASLFGPQTLTINIALCGDYAGLPSELEKTCPALVGDATCYTTYVINDGSTTYSQAYFEINYVNVYSSNPSSVTTISPSGPSTSATSTTASTSAAGTREGGATRPEQQLLLVAAGSLMSLFLFW

SEQ ID NO: 33 - protein, 463 aa, Cryptococcus sp.
MMFLSDKPNPICVIMDGKDQDATTDRGSLRFVFFRDTASPCLIGRQRIMLLSSQKDGYIKCRSWTDPLNTNLSTDVDLTPNRLANYPSASSWGDSLPLDRVLARDGFRFPPETYDNTTNGDTFWETAANTSLLYTTSAGTTILKVDNTTFVPYLEKRYAPKLLSKSAYDLGTVWVMDAVHMPYGCSVWPAFWTQGPSWPAGGEIDIIEGINLQPTNMIALHTSGNTSCTIPTTFTPSFSGRVSYPNCDNSQNFGSGCTVYDPNPNSYGQAFAEAGGGVFVAEFAEDGIRVWFMTRSAIPSTVRVNATQIDTSTLGIPVAEYPSSSCDITNLFGPQTLTINIALCGDYAGLPSELARTCPALVGDATCYTTYVINNASTTYAQAYFEINYINVYSSNPSTFTTISPSGPTPTSSTSTSTSTSASTPDAGTLSGGARRVERESVLVAMGLLVNFFHFGSILFGGE